SEQUENCE LISTING (101 sequences)

SEQ ID NO: 1 - DNA, 715 bp, Homo sapiens
tttcaagtttggaacaagcaaaagtgaagattgccaacacccaggaaataaggaagaatgagacagaaaccagatgtgttggtgtcatcctgtgactcagcttctattctggttgctgataaataaagaagagtttccaggtatgctatgtttggtttagccctaaactcggacagtaagtataccctaaagttaccaaaacagcactgctctgaaccacaaccatttcacatgtgttgatgggttaaaaagaaaaagaaaaaagaaatgaaaattggaaacatgacaacacaagatgaacacccatgggctttccacacagctggtaaatgtccctttgctctcagtggacacaggagctcttcttctgttttgacagcttttcctgccctgaaagaccctctttgggatcatttccccagtgggtttccccacctttccttcactcacatctctctctccccgactttcttcttcgaagttcccataggccaatctgtctttccaaccccaccccacatgcatgtacagacttcgtccgaagcctccctgtgagcaatatctttttctgagggcagtaaaccctgactcactgcctccttcctaccacagtttccaaaacactgctattgcgcccttgtctctatgcagatctcagtcagtctgggccaccatgtatgcaaacagctctttctgggaaatcccttcttgtctt

SEQ ID NO: 2 - DNA, 611 bp, Homo sapiens
tccagggcgatgtgggagcaggcattaaagtgggaggtttctgagaaagatattcactggctcagaacctcagttctcatcattgatacgagtacatcatagagcaattttgattcttcagggagaattaggtaatacatatgcttggaaattataagagaaatgaagacacgcctttgacattgtggttaagaaagctagaaaccacagcctctctgtcctttactgcctaataagaaaatcctttcctgattctatgaataattatttggacatggcagcaggagatgaggcctttccgttatgtgcagttcaaatacctatgtggtgcattcactatttgaaaccttcatgccaacatagctgccgggaggagtcatggagcctgcagatagaaatagccctgtccgctgggagaaagaggatcttggaggcccctcctctcatcctcttacacatactgatgaggaatgtttgcatctctgccagttgctatggagactaaatagggaacacatgtaaatattcggcaagcgtgagacacagcaaggtcctcctgcagtgtggttcctcttcttcttggctctttccctgatggctttgagctacag

SEQ ID NO: 3 - DNA, 604 bp, Homo sapiens
cacgcttcaggaaatggcacaaccaaaaagaagcctgggccaatatgaagccctcctagataagaaggggtttggaagctctgagaagattggtgagcattccaaagaatatggtttcaatgattggggtcaaccagcaataggagacacagataaaggtgaaagttagctcaggtaataatagcaccttgtagttttattcatgtgcttcagcctgaccagggaaggggtggggggtgacaaaacaaactcactttcacaccagcaagccaaggtgcagaggtcaagaggaggcagagggattattatcctaggtggttcacacagtatatcgaagcattagatagataaagccaatagcccaaggtcacacaattaggctttcactggttgggaattagagcagaatgtcctaatcttacccaggggctatttctagtagactctccatggagccttttgtttatacttcagctctatcttatcttgccttcctaggtagtgagcctactttttcccttggggacaagtagggaaatttgagtggttcagagtgcctggctagcttgagcttagaattcagctttctggacttggatgctct

SEQ ID NO: 4 - DNA, 24 bp, Mus musculus
gggcaggatgagtggttattgaga

SEQ ID NO: 5 - DNA, 27 bp, Homo sapiens
ggtgaaatgccctatagctcaactctg

SEQ ID NO: 6 - DNA, 24 bp, Mus musculus
gggtggaatacaaccacactgcat

SEQ ID NO: 7 - DNA, 26 bp, Homo sapiens
gtgcttatttcacattgcattcctgc

SEQ ID NO: 8 - DNA, 24 bp, Mus musculus
cactgtttgaaatgggtggcgatg

SEQ ID NO: 9 - DNA, 24 bp, Homo sapiens
tgctgcaatatagactttctgacc

SEQ ID NO: 10 - DNA, 24 bp, Mus musculus
gcatgaaaccacagagcctacaga

SEQ ID NO: 11 - DNA, 23 bp, Homo sapiens
gactgtcgtgtttgttctcactc

SEQ ID NO: 12 - DNA, 24 bp, Mus musculus
tcctacctgtctccagtgttgtga

SEQ ID NO: 13 - DNA, 24 bp, Homo sapiens
tgggcccagctcagtttatacctt

SEQ ID NO: 14 - DNA, 24 bp, Mus musculus
aacaacattccacagactggctcg

SEQ ID NO: 15 - DNA, 24 bp, Homo sapiens
ggtcccttcttcccatcagtttct

SEQ ID NO: 16 - DNA, 20 bp, Artificial
  Synthetic primer
tgccgctttgcaggtgtatt

SEQ ID NO: 17 - DNA, 21 bp, Artificial
  Synthetic primer
ccgatgctcagagctttctcc

SEQ ID NO: 18 - DNA, 23 bp, Artificial
  Synthetic primer
ctcctacccacgcagatttattc

SEQ ID NO: 19 - DNA, 21 bp, Artificial
  Synthetic primer
agagtgagaccacgaagagac

SEQ ID NO: 20 - DNA, 23 bp, Artificial
  Synthetic primer
catgagaagtatgacaacagcct

SEQ ID NO: 21 - DNA, 22 bp, Artificial
  Synthetic primer
agtccttccacgataccaaagt

SEQ ID NO: 22 - DNA, 111 bp, Artificial
  Synthetic probe
gattgccaacacccaggaaataaggaagaatgagacagaaaccagatgtgttggtgtcatcctgtgactcagcttctattctggttgctgataaataaagaagagtttcca

SEQ ID NO: 23 - DNA, 61 bp, Artificial
  Synthetic probe
ctgagggcagtaaaccctgactcactgcctccttcctaccacagtttccaaaacactgcta

SEQ ID NO: 24 - DNA, 86 bp, Artificial
  Synthetic probe
attgcgcccttgtctctatgcagatctcagtcagtctgggccaccatgtatgcaaacagctctttctgggaaatcccttcttgtct

SEQ ID NO: 25 - DNA, 141 bp, Artificial
  Synthetic probe
ctgagggcagtaaaccctgactcactgcctccttcctaccacagtttccaaaacactgctattgcgcccttgtctctatgcagatctcagtcagtctgggccaccatgtatgcaaacagctctttctgggaaatcccttct

SEQ ID NO: 26 - DNA, 46 bp, Artificial
  Synthetic probe
ccagtgggtttccccacctttccttcactcacatctctctctcccc

SEQ ID NO: 27 - DNA, 36 bp, Artificial
  Synthetic probe
ctccccgactttcttcttcgaagttcccataggcca

SEQ ID NO: 28 - DNA, 40 bp, Artificial
  Synthetic probe
catgcatgtacagacttcgtccgaagcctccctgtgagca

SEQ ID NO: 29 - DNA, 24 bp, Artificial
  Synthetic probe
tcatcctgtgactcagcttctatt

SEQ ID NO: 30 - DNA, 24 bp, Artificial
  Synthetic probe
gtaaaccctgactcactgcctcct

SEQ ID NO: 31 - DNA, 25 bp, Artificial
  Synthetic probe
ctctttctgggaaatcccttcttgt

SEQ ID NO: 32 - DNA, 29 bp, Artificial
  Synthetic probe
aacacccaggaaataaggaagaatgagac

SEQ ID NO: 33 - DNA, 21 bp, Artificial
  Synthetic probe
gttggtgtcatcctgtgactc

SEQ ID NO: 34 - DNA, 28 bp, Artificial
  Synthetic primer
ggtgtcatcctgtgagcttctattctgg

SEQ ID NO: 35 - DNA, 28 bp, Artificial
  Synthetic primer
ccagaatagaagctcacaggatgacacc

SEQ ID NO: 36 - DNA, 31 bp, Artificial
  Synthetic primer
ggcagtaaaccctcactgcctccttcctacc

SEQ ID NO: 37 - DNA, 31 bp, Artificial
  Synthetic primer
ggtaggaaggaggcagtgagggtttactgcc

SEQ ID NO: 38 - DNA, 29 bp, Artificial
  Synthetic primer
caaacagctctttctaatcccttcttgtc

SEQ ID NO: 39 - DNA, 29 bp, Artificial
  Synthetic primer
gacaagaagggattagaaagagctgtttg

SEQ ID NO: 40 - DNA, 34 bp, Artificial
  Synthetic primer
ccatgggctttccacatggtaaatgtccctttgc

SEQ ID NO: 41 - DNA, 33 bp, Artificial
  Synthetic primer
gcaaagggacatttaccatgtggaaagcccatg

SEQ ID NO: 42 - DNA, 24 bp, Artificial
  Synthetic primer
aggtgagcggatatcaaccaagga

SEQ ID NO: 43 - DNA, 24 bp, Artificial
  Synthetic primer
agaactcagtgccgtgtcgatagt

SEQ ID NO: 44 - DNA, 27 bp, Artificial
  Synthetic primer
ccaggtatgctatgtttggttaagccc

SEQ ID NO: 45 - DNA, 24 bp, Artificial
  Synthetic primer
gtggagttggaaagacagattggc

SEQ ID NO: 46 - DNA, 24 bp, Artificial
  Synthetic primer
tctctcccactgctttcctccaaa

SEQ ID NO: 47 - DNA, 26 bp, Artificial
  Synthetic primer
gtgcttatttcacattgcattcctgc

SEQ ID NO: 48 - DNA, 21 bp, Homo sapiens
cagaaaccagatgtgttggtg

SEQ ID NO: 49 - DNA, 13 bp, Homo sapiens
aacacatctggtt

SEQ ID NO: 50 - DNA, 23 bp, Homo sapiens
tagaagctgagtcacaggatgac

SEQ ID NO: 51 - DNA, 21 bp, Homo sapiens
agaagctgagtcacaggatga

SEQ ID NO: 52 - DNA, 17 bp, Homo sapiens
aagctgagtcacaggat

SEQ ID NO: 53 - DNA, 21 bp, Homo sapiens
atcctgtgactcagcttctat

SEQ ID NO: 54 - DNA, 11 bp, Homo sapiens
tgtgactcagc

SEQ ID NO: 55 - DNA, 11 bp, Homo sapiens
gctgagtcaca

SEQ ID NO: 56 - DNA, 13 bp, Homo sapiens
tgctgataaataa

SEQ ID NO: 57 - DNA, 17 bp, Homo sapiens
ttctttatttatcagca

SEQ ID NO: 58 - DNA, 19 bp, Homo sapiens
gaagagtttccaggtatgc

SEQ ID NO: 59 - DNA, 17 bp, Homo sapiens
gcatacctggaaactct

SEQ ID NO: 60 - DNA, 19 bp, Homo sapiens
tttcttcttcgaagttccc

SEQ ID NO: 61 - DNA, 15 bp, Homo sapiens
taaaccaaacatagc

SEQ ID NO: 62 - DNA, 15 bp, Homo sapiens
gacagtaagtatacc

SEQ ID NO: 63 - DNA, 19 bp, Homo sapiens
tataccctaaagttaccaa

SEQ ID NO: 64 - DNA, 15 bp, Homo sapiens
ggttgtggttcagag

SEQ ID NO: 65 - DNA, 13 bp, Homo sapiens
tcaacacatgtga

SEQ ID NO: 66 - DNA, 21 bp, Homo sapiens
aaaagaaaaagaaaaaagaaa

SEQ ID NO: 67 - DNA, 21 bp, Homo sapiens
aaaagaaatgaaaattggaaa

SEQ ID NO: 68 - DNA, 15 bp, Homo sapiens
gaaatgaaaattgga

SEQ ID NO: 69 - DNA, 15 bp, Homo sapiens
cctatgggaacttcg

SEQ ID NO: 70 - DNA, 13 bp, Homo sapiens
cagattggcctat

SEQ ID NO: 71 - DNA, 15 bp, Homo sapiens
taggccaatctgtct

SEQ ID NO: 72 - DNA, 15 bp, Homo sapiens
tgtggggtggggttg

SEQ ID NO: 73 - DNA, 23 bp, Homo sapiens
gccctcagaaaaagatattgctc

SEQ ID NO: 74 - DNA, 21 bp, Homo sapiens
aggcagtgagtcagggtttac

SEQ ID NO: 75 - DNA, 21 bp, Homo sapiens
aaaccctgactcactgcctcc

SEQ ID NO: 76 - DNA, 21 bp, Homo sapiens
aaaccctgactcactgcctcc

SEQ ID NO: 77 - DNA, 11 bp, Homo sapiens
gagtcagggtt

SEQ ID NO: 78 - DNA, 11 bp, Homo sapiens
cctgactcact

SEQ ID NO: 79 - DNA, 11 bp, Homo sapiens
agtgagtcagg

SEQ ID NO: 80 - DNA, 11 bp, Homo sapiens
ggaaggaggca

SEQ ID NO: 81 - DNA, 15 bp, Homo sapiens
aactgtggtaggaag

SEQ ID NO: 82 - DNA, 27 bp, Homo sapiens
cagtgttttggaaactgtggtaggaag

SEQ ID NO: 83 - DNA, 17 bp, Homo sapiens
tctatgcagatctcagt

SEQ ID NO: 84 - DNA, 17 bp, Homo sapiens
gatctgcatagagacaa

SEQ ID NO: 85 - DNA, 17 bp, Homo sapiens
tgtatgcaaacagctct

SEQ ID NO: 86 - DNA, 13 bp, Homo sapiens
ctgggaaatccct

SEQ ID NO: 87 - DNA, 13 bp, Homo sapiens
aagggatttccca

SEQ ID NO: 88 - DNA, 17 bp, Homo sapiens
aagacaagaagggattt

SEQ ID NO: 89 - DNA, 717 bp, Homo sapiens
agagagtgagagatcgaaagatgaggagggaatcacccatcaagagtctgtttatgtacatcgtggccccgacctaccgagatctgcatagagacaagagtgaaataagactgtgttttggaaactgtggtaagaaggaggcaatgagtcagtgcttaccatccccagaaaaagatattgctcactggggagtctttggggcaaagtctgtacatgtatctgggggtggagttggaaagacagattggcatatgggtatttggaggaaagcagtgggagagagagagaatgtgaatgaatgaaaagtgggaaaacacattggggaaaacacatagtaccgaaagcgggtctctgaggcagaaggagccatcaaagcggaagcagaactcagtgccgtgtcgatagtgaagggagatttgaagattgcatagggaacccagagacattcatttcatttttttcatgcttctaattttaatttcttttcagctcaacacgtgaaataattgtggtttaaagcaatgtagtttcggtaaccttagggctatacttatgtccaagtttagggcttaaccaaacatagcatacctggaaatggtcctttatttatcagtagctgaaatagaagctgagtcacaggagcgacacaaacacatctggtgtctgtctcatgccttcaaatgccacagggtgttggcgatctttgcttttacttgc

SEQ ID NO: 90 - DNA, 727 bp, Homo sapiens
ggtatttcccagtggctcatggtaaatgagttccttaaaaatgagccacgctgaggcctgaaactacagggcttactgatgtggctgcaagctgcagctctaagccagaaaggggacaagctaaggagaagaggaaccacctttgcactggcggacttcagcgtgtctcacgcatgccgaatgtttacatgtgttccccacttggtatccatagcaactggcagaggtgcaaaaatgcttcattggtatttgtaagaggatgagaagaaagccctccaagatcctctttctcccagtgggcagggctatttccatccatggcttcgtgacttctcccagcagccgtttggcatgaggatttcaaacagcagggagccagtgagtccaccggataggtatttgaaatgtactgaatggaaagccctcatctcctgctccctcttccaggcaattattcatggagtcaggagaggattctgttattcaaggagtctccatagtaaggggctgagagattgtggtttctagtctttaaccacaacatttaaggcgtggctccatttttcttataatttccaagtgtacatcattacctaattctccctgaagaatcgaaattgctttatgatgcatatgtattagtgatgtgagctgaggttcggagccagcaaacatctttctcagaaacctccctcttgttctcctctcccatgttgccccttgaagt

SEQ ID NO: 91 - DNA, 567 bp, Homo sapiens
gctcaggctggccagctggctcactctgggccactcaagtttcccactactcatccccaagggaaaaagtaggctagctacctgggcaggtaagataagatagaaccaaggtataaacaaaaggctccatcaaaagtctaccagaaatagcccctgggtaagattaggacactctgctctaattcccaccctgtgaaagcctaattgtttgaccttgggctattggctttatctgtctaatggttcaatatactgtgtgaaccatttggggataataatccctctgcctcccctagacctctccacctttgcctgctggtgtgaaagtgttttgtgctagtaggcagaaacacgtgatataaaactacaaggtcctattattacctaggataactttcgcctctatctatgtctcctgttgctggttgaccccaatcattgaaactgtattctttggaatgctcagcagtctcctccaagctgccattccctccttatctaggtgggcccagtattggctcaggcttcttggttgtgccacttcctaaggcttgcaaatgcattcag

SEQ ID NO: 92 - DNA, 21 bp, Homo sapiens
aaaccctgactcactgcctcc

SEQ ID NO: 93 - DNA, 13 bp, Homo sapiens
cagtgagtcaggg

SEQ ID NO: 94 - DNA, 13 bp, Homo sapiens
ccctgactcactg

SEQ ID NO: 95 - DNA, 21 bp, Homo sapiens
aggcagtgagtcagggtttac

SEQ ID NO: 96 - DNA, 21 bp, Homo sapiens
atcctgtgactcagcttctat

SEQ ID NO: 97 - DNA, 13 bp, Homo sapiens
agctgagtcacag

SEQ ID NO: 98 - DNA, 13 bp, Homo sapiens
ctgtgactcagct

SEQ ID NO: 99 - DNA, 21 bp, Homo sapiens
agaagctgagtcacaggatga

SEQ ID NO: 100 - DNA, 15 bp, Homo sapiens
ctgggaaatcccttc

SEQ ID NO: 101 - DNA, 15 bp, Homo sapiens
aagggatttcccaga

(19) United States
(12) Patent Application Publication
(10) Pub. No.: US 2013/0029346 A1
(43) Pub. Date: Jan. 31, 2013

(54) VECTOR AND SCREENING ASSAY FOR CD44 EXPRESSING CARCINOMAS

(71) Applicant: Li Cai, Warren, NJ (US)

(72) Inventor: Li Cai, Warren, NJ (US)

(73) Assignee: RUTGERS, THE STATE UNIVERSITY OF NEW JERSEY, New Brunswick, NJ (US)

(21) Appl. No.: 13/561,908

(22) Filed: Jul. 30, 2012

Related U.S. Application Data
(60) Provisional application No. 61/513,555, filed on Jul. 30, 2011.

(51) Int. Cl. C12Q 1/68; G01N 21/76; C12N 15/63; G01N 21/64

(52) U.S. Cl. 435/6.13; 435/320.1

(57) ABSTRACT

The present invention relates, in part, to the discovery of cis-regulatory regions for the expression of CD44 in normal cells and/or and over-expression in cancer cells or cancer stem cells. To this end, the present invention provides isolated DNA, vectors, kits, and methods that may be used for the evaluation and/or screening one or more therapeutic agents for the treatment of a CD44 expressing carcinoma.

### CROSS REFERENCES TO RELATED APPLICATIONS

The present application claims priority to U.S. Provisional Patent Application No. 61/513,555, filed on Jul. 30, 2011, the contents of which is incorporated herein by reference in its entirety.

### STATEMENT REGARDING FEDERALLY FUNDED RESEARCH

This invention was made with government support under Grant CA 133675 awarded by the National Institutes of Health. Accordingly, the U.S. Government has certain rights in this invention.

### FIELD OF THE INVENTION

The present invention relates to novel cis-elements that direct CD44 expression in normal cells, cancer cells, and cancer stem cells, and includes the isolated nucleic acid thereof, vectors thereof, kits, and related methods of use.

### BACKGROUND OF THE INVENTION

Breast cancer remains the most common form of cancer among women and the second leading cause of cancer related deaths. Recently, a small subset of cancer cells was identified by cell surface markers (e.g., up-regulation of CD44 and down-regulation of CD24) as cancer stem cells (CSCs). This CD44⁺/CD24^{low/-} signature is observed in other CSCs including prostate, pancreatic, brain and leukemia stem cells. In addition to stem cell characteristics (i.e., the ability to self-renew and differentiate into all cell types in a mammary gland), CSCs are resistant to chemotherapy- and radiation treatment, and have the increased ability to metastasize and develop new tumors throughout the body.

As a cell surface glycoprotein, CD44 is ubiquitously expressed on most cells throughout the body. CD44 is involved in cellular processes including cell-cell and cell-extracellular matrix adhesion, migration, differentiation and survival, all of which make CD44 pro-oncogenic by nature. Studies have established that CD44 is a therapeutic target for metastastic tumors. By targeting CD44, human acute myeloid leukemic stem cells can be eradicated. In addition, directly repressing CD44 expression by miR-34a inhibits prostate CSCs and metastasis.

Over-expression of CD44 has been correlated to a number of transcription factors (TFs) including Egr1, AP-1, NFκB, and c/EBPβ. Most notably, AP-1 and NFκB have been shown to directly correlate with CD44, by binding the CD44 promoter. AP-1, a leucine zipper TF consists of two families, Jun (c-Jun, JunB and JunD) and Fos (c-Fos, FosB, Fra1 and Fra2). The Jun proteins can form homodimers with one another or heterodimers with the Fos proteins. Together these proteins bind to core sequences in the genome to regulate expression of a target gene. AP-1 is involved in a number of cellular process similar to CD44 including differentiation, proliferation and apoptosis. Regulation by AP-1 is induced by growth factors, cytokines and oncoproteins, which are implicated in the proliferation and survival of cells. AP-1 activity in a cell, whether it be pro-apoptotic or pro-oncogenic, is determined by the composition of the homodimer or heterodimer formed as well as the tumor type and state of differentiation of the cell.

NFκB, like AP-1, has been linked to the up-regulation of CD44, but no direct evidence has been shown. Increased HGF has been shown to enhance expression of CD44 through a complex of NFκB, c/EBPβ and EGR1. NFκB proteins have also been shown to be up-regulated in breast cancer stem cells (BCSCs), and their expressions have been correlated to increased expression of tumor stem cell markers, including CD44. Interestingly, the reduction of NFκB in a murine cell line Met-1 was able to reduce the number of CD44⁺/CD24^{-/low} cells.

Despite intense research on CD44, the mechanism by which the protein is up-regulated in cancer and BCSCs is not well understood. Gene regulatory elements, e.g., promoters and enhancers, recruit TFs and chromatin modifying proteins, and allow transcription of the target genes to occur. Enhancers are required for both temporal and tissue/cell specific gene expression. Therefore, it is an important task to identify and understand their role in gene expression of both normal and pathological conditions.

### SUMMARY OF THE INVENTION

The present invention relates, in part, to the discovery of cis-regulatory regions for the expression of CD44 in normal cells and/or the over-expression of CD44 in cancer cells or CSCs. More specifically, it is demonstrated herein that certain non-coding CD44 regulatory regions have the ability to drive CD44 expression through an interaction with trans-acting factors, such as AP-1 and/or NFκB. These CD44 regulatory regions provide a target for the treatment of cancer, particularly cancers exhibiting high CD44 expression levels. To this end, the present invention provides isolated DNA, vectors, kits, and methods for evaluating and/or screening one or more potential therapeutic agents for the treatment of a CD44 expressing carcinoma.

In one aspect, the present invention relates to a method for identifying a compound or therapeutic agent that inhibits CD44 expression in a cell, by (a) providing a cell that expresses a gene using a CD44 regulatory region; (b) contacting the cell with a compound or therapeutic agent; and (c) detecting a change in expression level of the gene. The CD44 regulatory region, in certain aspects, includes a sequence selected from the group consisting of SEQ ID NO.: 1 (CR1), SEQ ID NO.: 89 (CR1), SEQ ID NO.: 2 (CR2), SEQ ID NO.: 90 (CR2), SEQ ID NO.: 3 (CR3), SEQ ID NO.: 91 (CR3), combinations thereof, and variants thereof. In further aspects, the CD44 regulatory region comprises a binding region for a factor selected from the group consisting of AP-1, NFκB, a combination thereof, and variants thereof. The AP-1 binding region may include any one or combination of SEQ ID NOS.: 92-99 or a variant thereof, and the NFκB binding region comprises any one of or combination of SEQ ID NOS.: 100-101 or a variant thereof.

The gene used in the foregoing method may include CD44 that is natively or artificially expressed in a cell. Alternatively, the gene may include a non-CD44 coding region, such as, but not limited to, that of a reporter protein, which is transfected into a cell. Reporter proteins may include, but are not limited to, green fluorescent protein, red fluorescent protein, yellow fluorescent protein, beta-galactosidase, luciferase, and combinations thereof, or any other reporter protein discussed herein or otherwise known in the art.

Methods of detecting protein levels may include any method known in the art. Such methods may include, but are not limited to, an ELISA assay, a radioimmunassay, a Western blot analysis, flow cytometry, a high content screening assay or any other detection method discussed herein or otherwise known in the art.

In further embodiments, the present invention relates to a vector that includes a gene; a promoter region; and a non-coding CD44 regulatory region that controls expression of the gene. The non-coding CD44 regulatory region may include any of the CR1-CR3 sequences identified above or otherwise herein. It may also, or alternatively, include an AP-1 binding region, a NFκB binding region, a combination thereof, and variants thereof (as defined herein). The gene expressed may include a CD44 coding region or a non-CD44 coding region, such as the reporter proteins identified herein or otherwise known in the art.

In even further embodiments, the present invention relates to a kit for identifying a compound or therapeutic agent that inhibits CD44 expression in a cell, including a vector comprising a reporter gene; a promoter region; and a non-coding CD44 regulatory region that controls expression of the reporter gene; and a reagent for detecting a product of the reporter gene.

Additional embodiments and advantages to the present invention will be readily apparent to one of skill in the art, based at least on the disclosure and Examples provided herein.

To aid in the understanding of the invention, the following non-limiting definitions are provided:

The term “AP-1,” as used herein, has a standard meaning understood in the art. It is a leucine zipper transcription factor that is a heterodimeric protein composed of proteins within the Fox and Jun families.

The term “CD44” also has a standard meaning understood in the art. The full length CD44 protein occurs in nature in several variants, with the human variant having 742 amino acids in length.

As used herein, the term “contacting” refers to directly or indirectly causing placement together of moieties, such that the moieties directly or indirectly come into physical association with each other, whereby a desired outcome is achieved. Contacting may occur, for example, in any number of buffers, salts, solutions, or in a cell or cell extract. Thus, as used herein, one can “contact” a target cell with a therapeutic agent as disclosed herein even though the therapeutic agent and cell do not necessarily physically join together (as, for example, is the case where a ligand and a receptor physically join together), as long as the desired outcome is achieved (e.g., reduced activity of the CD44 regulatory region). Contacting thus includes acts such as placing moieties together in a container (e.g., adding a compound as disclosed herein to a container comprising cells for in vitro studies) as well as administration of the compound to a target entity (e.g., injecting a compound as disclosed herein into a laboratory animal for in vivo testing, or into a human for therapy or treatment purposes).

As used herein, “measure” or “determine” refers to any qualitative or quantitative determinations.

The term “NFκB,” as used herein, also has a standard meaning understood in the art. It is a transcription factor that is a heterodimeric protein composed of proteins Rel family.

As used herein, the terms “peptide,” “polypeptide” and “protein” all refer to a primary sequence of amino acids that are joined by covalent “peptide linkages.” In general, a peptide consists of a few amino acids, typically from 2-50 amino acids, and is shorter than a protein. The term “polypeptide” encompasses peptides and proteins. In some embodiments, the peptide, polypeptide or protein is synthetic, while in other embodiments, the peptide, polypeptide or protein is recombinant or naturally occurring.

As used herein, the terms “reduce” or “reduction,” particularly when used in the context of a screening assay, refer to a comparative decrease in a specified response of a designated material (e.g., expression, enzymatic activity) in the presence of a specified reagent or therapeutic agent.

As used herein, “therapeutic agent,” “potential therapeutic agent,” or “test compound” refers to any purified molecule, substantially purified molecule, molecules that are one or more components of a mixture of molecules, or a mixture of a molecules with any other material that can be analyzed using the methods of the present invention. Such agents can be organic or inorganic chemicals, or biomolecules, and all fragments, analogs, homologs, conjugates, and derivatives thereof. Biomolecules include proteins, polypeptides, nucleic acids, lipids, polysaccharides, and all fragments, analogs, homologs, conjugates, and derivatives thereof. These agents can be of natural or synthetic origin, and can be isolated or purified from their naturally occurring sources, or can be synthesized de novo. These agents can be defined in terms of structure or composition, or can be undefined. The agent can be an isolated product of unknown structure, a mixture of several known products, or an undefined composition comprising one or more compounds.

### BRIEF DESCRIPTION OF THE FIGURES

FIG. 1 illustrates the prediction of cis-regulatory elements for CD44 expression using sequence alignment analysis, with FIG. 1A providing a genomic map of human CD44 and surrounding genes located on chromosome 11p13; FIG. 1B providing multiple sequence alignment of homologous CD44 sequences using human sequence as baseline; and FIG. 1C illustrating the plasmid reporter construct containing a conserved region of CD44, a minimal beta-globin-promoter (βGP), and green fluorescent protein (GFP).

FIG. 2 illustrates the results of the conserved region tests for the ability to direct reporter gene expression in transfected breast cancer cell lines. FIGS. 2A-C illustrate GFP expression in all three cell lines from transfection of a positive control construct (CAG-GFP); FIGS. 2D-F illustrate no GFP expression from transfection of a negative control; FIGS. 2G-I illustrate GFP expression from transfection of a CD44CR1-βGP-GFP construct in breast cancer cell lines; FIG. 2J illustrates quantification of GFP expression.

FIG. 3 illustrates the results of an electrophoresis mobility shift assay (EMSA) performed to determine the in vitro binding activities of nuclear protein factors with CD44CR1. FIG. 3A illustrates DNA probe design using mouse sequence and TFBSs within each probe; FIG. 3B illustrates no band shift with the AP-1-1 probe in any of the three cell lines; FIG. 3C illustrates a band shift in all three cell lines with the AP-1-2 probe; FIG. 3D illustrates that the NFκB probe showed a band shift that was successfully competed away in all three cell lines; FIG. 3E illustrates that the mutant competition probe for NFκB was able to successfully compete away the band shift in all three cell lines (arrowhead); FIG. 3F illustrates the supershift with NF-κB antibodies performed with SUM159 nuclear extract.

FIG. 4 illustrates that the mutation of AP-1 and NFκB binding sites in CR1 reduces reporter GFP expression. FIGS. 4A-I provide a schematic of each mutation of CD44CR1-GFP construct; FIGS. 4A′-I′ provide images of SUM159 cells transfected with mutated constructs; FIG. 4J provides the number of GFP-expressing cells/total number of cells counted.

FIG. 5 illustrates that AP-1 and NFκB expression differs among breast cancer cell lines FIGS. 5A-C illustrates that cells were stained with cJun antibody reveal normal TF expression in all three cell lines; FIGS. 5D-F illustrates that cRel expression was normal in SUM159 and MCF7 cell lines but was not expressed in MDA-MB-231 (5D and 5D′); FIGS. 5G-I illustrated that JunB expression was normal in SUM159 and MCF7 cell lines but was not expressed in MDA-MB-231.

FIG. 6 illustrates differential AP-1 factor binding to CD44CR1 in breast cancer cell lines SUM159 and MCF7. ChIP with AP-1 antibodies resulted in amplification of a region of CR1 with inverted repeat AP-1 binding sites. Representative results of at least two independent immunoprecipitation experiments and multiple independent PCR analyses are shown. Strong PCR amplification of CR1 region with JunB binding was seen in SUM159 cells and with JunD binding in MCF7 cells.

FIG. 7 illustrates CD44 and CD24 expression in breast cancer cell lines as detected by immunocytochemistry. Human cell lines MDA-MB-231 (FIGS. 7A-A′″), SUM159 (FIGS. 7B-B′″), and MCF7 (FIGS. 7C-C′″) were fixed and stained for CD44 and CD24, nuclei were stained with Hoechst33342; FIG. D illustrates real-time PCR analysis of CD44 and CD24 mRNA levels in breast cancer cell lines.

FIG. 8 illustrates genomic sequence alignment of conserved regions and no mutations in the transcription factor binding sites (TFBSs). Genomic DNA was obtained from the cell lines MDA-MB-231, SUM159 and MCF7, and was sequenced at CD44CR1 conserved region and aligned using ClustalW. Alignment of CD44CR1 sequences identified a 5 bp deletion located in SUM159 genomic DNA. However, these mutations do not change TFBSs.

FIG. 9 illustrates an EMSA to identify protein factors that bind with CD44CR1 in breast cancer cell lines using three probes designed to cover the conserved region of CD44CR1, Probe 1 (FIG. 9A) identified binding in MDA-MB-231 and MCF2; Probe 2 (FIG. 9B) showed strong binding present in all three cell lines; and Probe 3 (FIG. 9C) showed multiple shifted bands and was successfully competed away in all three cell lines using unlabeled probes.

FIG. 10 provides an analysis of AP-1 and NF-κB binding sites using site directed mutagenesis of CD44CR1 in MDA-MB-231 cells. FIG. 10A provides transfection of a positive control construct CAG-GFP; FIG. 10B of CD44CR1; FIG. 10C of a Control-GFP, which shows little different in GFP expression when compared to CD44CR1; FIGS. 10D-F provide that no GFP expression was detected with a single site mutation of AP-1-1, AP-1-2 and NF-κB.

FIG. 11 provides an analysis of AP-1 and NF-κB binding sites using site directed mutagenesis of CD44CR1 in MCF7 cells. FIG. 11A provides transfection of positive control CAG-GFP; FIG. 11B of CD44CR1-GFP; and FIG. 11C of a Control-GFP, which showed little difference in GFP expression when compare to CD44CR1; FIGS. 11D-F provide no GFP expression was detected with a single site mutation of AP-1-1, AP-1-2, and NF-κB.

FIG. 12 illustrates that AP-1 factors cFos (FIGS. 12A-C), Fra1 (FIGS. 12D-F) and Fra2 (FIGS. G-I) show normal expression in breast cancer cell lines MDA-MB-231, SUM159 and MCF7. (MDA-MB-231, SUM159 and MCF7 cell lines were co-stained with antibodies corresponding to CD44 and the AP-1 TFs: cFos (FIGS. 12A-C), Fra1 (FIGS. 12D-F) and Fra2 (FIGS. 12G-I). Staining with cFos, Fra1 and Fra2 revealed normal TF expression in all three cell lines. Nuclei were stained with Hoechst33342. Scale bar=50 μM)

FIG. 13 illustrates that NF-κB factors p50 (FIGS. 13A-C) and p65 (13D-F) show normal expression in cancer cell lines MDA-MB-231, SUM159 and MCF7. (CD44 staining in MDA-MB-231, SUM159 and MCF7 cell lines were co-stained with antibodies NFκB TFs p50 (FIGS. 13A-C) and p65 (FIGS. 13D-F). Staining with p50 and p65 revealed normal TF expression in all three cell lines. Nuclei were stained with Hoechst33342. (Scale bar=50 μM.)

FIG. 14 illustrates that NF-κB factors cRel, p50 and p65 do not bind to CD44CR1 by ChIP in either SUM159 or MCF7 cell lines.

### DETAILED DESCRIPTION OF PREFERRED EMBODIMENTS

In one aspect, the present invention relates to the isolation, identification and characterization of non-coding regulatory regions of the CD44 gene located in its intronic region, i.e. the 5′ or 3′ intergenic regions. These regulatory regions are shown below to have a unique ability to direct the expression of the CD44 gene in a cell-type specific manner, particularly, though not exclusively, in breast cancer stem cells or stem-like cells. Expression is further shown to be dependent upon the presence and binding of trans-acting factors AP-1 and NFκB within the cell. Expression levels may be normal or consistent with a level detected in a non-cancerous cell. However, in certain embodiments, CD44 expression levels are above a normal or baseline level, which are or may be consistent with the heighted expression levels observed in certain cancer cells or cancer stem cells. These CD44 regulatory regions provide a potential target for the treatment of cancer, particularly cancers exhibiting high CD44 expression levels. To this end, the present invention provides isolated DNA, vectors, kits and methods for evaluating and/or screening one or more potential therapeutic agents for the treatment of a CD44 expressing carcinoma.

In certain embodiments, the CD44 regulatory regions of the present invention refer to the sequences identified herein as conservative regions (CR) CR1, CR2, and CR3. These regions, in certain embodiments, have the following DNA sequence:

Mouse CD44CR1, non-coding DNA
(SEQ ID NO.: 1)
TTTCAAGTTTGGAACAAGCAAAAGTGAAGATTGCCAACA
CCCAGGAAATAAGGAAGAATGAGACAGAAACCAGATGT
GTTGGTGTCATCCTGTGACTCAGCTTCTATTCTGGTTGCT
GATAAATAAAGAAGAGTTTCCAGGTATGCTATGTTTGGT
TTAGCCCTAAACTCGGACAGTAAGTATACCCTAAAGTTA
CCAAAACAGCACTGCTCTGAACCACAACCATTTCACATG
TGTTGATGGGTTAAAAAGAAAAAGAAAAAAGAAATGAA
AATTGGAAACATGACAACACAAGATGAACACCCATGGG
CTTTCCACACAGCTGGTAAATGTCCCTTTGCTCTCAGTGG
ACACAGGAGCTCTTCTTCTGTTTTGACAGCTTTTCCTGCC
CTGAAAGACCCTCTTTGGGATCATTTCCCCAGTGGGTTTC
CCCACCTTTCCTTCACTCACATCTCTCTCTCCCCGACTTTC
TTCTTCGAAGTTCCCATAGGCCAATCTGTCTTTCCAACCC
CACCCCACATGCATGTACAGACTTCGTCCGAAGCCTCCC
TGTGAGCAATATCTTTTTCTGAGGGCAGTAAACCCTGACT
CACTGCCTCCTTCCTACCACAGTTTCCAAAACACTGCTAT
TGCGCCCTTGTCTCTATGCAGATCTCAGTCAGTCTGGGCC
ACCATGTATGCAAACAGCTCTTTCTGGGAAATCCCTTCTT
GTCTT
Mouse CD44CR2, non-coding DNA
(SEQ ID NO.: 2)
TCCAGGGCGATGTGGGAGCAGGCATTAAAGTGGGAGGTT
TCTGAGAAAGATATTCACTGGCTCAGAACCTCAGTTCTC
ATCATTGATACGAGTACATCATAGAGCAATTTTGATTCTT
CAGGGAGAATTAGGTAATACATATGCTTGGAAATTATAA
GAGAAATGAAGACACGCCTTTGACATTGTGGTTAAGAAA
GCTAGAAACCACAGCCTCTCTGTCCTTTACTGCCTAATAA
GAAAATCCTTTCCTGATTCTATGAATAATTATTTGGACAT
GGCAGCAGGAGATGAGGCCTTTCCGTTATGTGCAGTTCA
AATACCTATGTGGTGCATTCACTATTTGAAACCTTCATGC
CAACATAGCTGCCGGGAGGAGTCATGGAGCCTGCAGATA
GAAATAGCCCTGTCCGCTGGGAGAAAGAGGATCTTGGAG
GCCCCTCCTCTCATCCTCTTACACATACTGATGAGGAATG
TTTGCATCTCTGCCAGTTGCTATGGAGACTAAATAGGGA
ACACATGTAAATATTCGGCAAGCGTGAGACACAGCAAG
GTCCTCCTGCAGTGTGGTTCCTCTTCTTCTTGGCTCTTTCC
CTGATGGCTTTGAGCTACAG
Mouse CD44CR3, non-coding DNA
(SEQ ID NO.: 3)
CACGCTTCAGGAAATGGCACAACCAAAAAGAAGCCTGG
GCCAATATGAAGCCCTCCTAGATAAGAAGGGGTTTGGAA
GCTCTGAGAAGATTGGTGAGCATTCCAAAGAATATGGTT
TCAATGATTGGGGTCAACCAGCAATAGGAGACACAGATA
AAGGTGAAAGTTAGCTCAGGTAATAATAGCACCTTGTAG
TTTTATTCATGTGCTTCAGCCTGACCAGGGAAGGGGTGG
GGGGTGACAAAACAAACTCACTTTCACACCAGCAAGCCA
AGGTGCAGAGGTCAAGAGGAGGCAGAGGGATTATTATC
CTAGGTGGTTCACACAGTATATCGAAGCATTAGATAGAT
AAAGCCAATAGCCCAAGGTCACACAATTAGGCTTTCACT
GGTTGGGAATTAGAGCAGAATGTCCTAATCTTACCCAGG
GGCTATTTCTAGTAGACTCTCCATGGAGCCTTTTGTTTAT
ACTTCAGCTCTATCTTATCTTGCCTTCCTAGGTAGTGAGC
CTACTTTTTCCCTTGGGGACAAGTAGGGAAATTTGAGTG
GTTCAGAGTGCCTGGCTAGCTTGAGCTTAGAATTCAGCTT
TCTGGACTTGGATGCTCT
Human CD44CR1, non-coding DNA
(SEQ ID NO.: 89)
AGAGAGTGAGAGATCGAAAGATGAGGAGGGAATCACCC
ATCAAGAGTCTGTTTATGTACATCGTGGCCCCGACCTACC
GAGATCTGCATAGAGACAAGAGTGAAATAAGACTGTGTT
TTGGAAACTGTGGTAAGAAGGAGGCAATGAGTCAGTGCT
TACCATCCCCAGAAAAAGATATTGCTCACTGGGGAGTCT
TTGGGGCAAAGTCTGTACATGTATCTGGGGGTGGAGTTG
GAAAGACAGATTGGCATATGGGTATTTGGAGGAAAGCA
GTGGGAGAGAGAGAGAATGTGAATGAATGAAAAGTGGG
AAAACACATTGGGGAAAACACATAGTACCGAAAGCGGG
TCTCTGAGGCAGAAGGAGCCATCAAAGCGGAAGCAGAA
CTCAGTGCCGTGTCGATAGTGAAGGGAGATTTGAAGATT
GCATAGGGAACCCAGAGACATTCATTTCATTTTTTTCATG
CTTCTAATTTTAATTTCTTTTCAGCTCAACACGTGAAATA
ATTGTGGTTTAAAGCAATGTAGTTTCGGTAACCTTAGGG
CTATACTTATGTCCAAGTTTAGGGCTTAACCAAACATAG
CATACCTGGAAATGGTCCTTTATTTATCAGTAGCTGAAAT
AGAAGCTGAGTCACAGGAGCGACACAAACACATCTGGT
GTCTGTCTCATGCCTTCAAATGCCACAGGGTGTTGGCGAT
CTTTGCTTTTACTTGC
Human CD44CR2, non-coding
(SEQ ID NO.: 90)
GGTATTTCCCAGTGGCTCATGGTAAATGAGTTCCTTAAA
AATGAGCCACGCTGAGGCCTGAAACTACAGGGCTTACTG
ATGTGGCTGCAAGCTGCAGCTCTAAGCCAGAAAGGGGAC
AAGCTAAGGAGAAGAGGAACCACCTTTGCACTGGCGGA
CTTCAGCGTGTCTCACGCATGCCGAATGTTTACATGTGTT
CCCCACTTGGTATCCATAGCAACTGGCAGAGGTGCAAAA
ATGCTTCATTGGTATTTGTAAGAGGATGAGAAGAAAGCC
CTCCAAGATCCTCTTTCTCCCAGTGGGCAGGGCTATTTCC
ATCCATGGCTTCGTGACTTCTCCCAGCAGCCGTTTGGCAT
GAGGATTTCAAACAGCAGGGAGCCAGTGAGTCCACCGG
ATAGGTATTTGAAATGTACTGAATGGAAAGCCCTCATCT
CCTGCTCCCTCTTCCAGGCAATTATTCATGGAGTCAGGAG
AGGATTCTGTTATTCAAGGAGTCTCCATAGTAAGGGGCT
GAGAGATTGTGGTTTCTAGTCTTTAACCACAACATTTAAG
GCGTGGCTCCATTTTTCTTATAATTTCCAAGTGTACATCA
TTACCTAATTCTCCCTGAAGAATCGAAATTGCTTTATGAT
GCATATGTATTAGTGATGTGAGCTGAGGTTCGGAGCCAG
CAAACATCTTTCTCAGAAACCTCCCTCTTGTTCTCCTCTC
CCATGTTGCCCCTTGAAGT
Human CD44CR3, non-coding DNA
(SEQ ID NO.: 91)
GCTCAGGCTGGCCAGCTGGCTCACTCTGGGCCACTCAAG
TTTCCCACTACTCATCCCCAAGGGAAAAAGTAGGCTAGC
TACCTGGGCAGGTAAGATAAGATAGAACCAAGGTATAA
ACAAAAGGCTCCATCAAAAGTCTACCAGAAATAGCCCCT
GGGTAAGATTAGGACACTCTGCTCTAATTCCCACCCTGT
GAAAGCCTAATTGTTTGACCTTGGGCTATTGGCTTTATCT
GTCTAATGGTTCAATATACTGTGTGAACCATTTGGGGAT
AATAATCCCTCTGCCTCCCCTAGACCTCTCCACCTTTGCC
TGCTGGTGTGAAAGTGTTTTGTGCTAGTAGGCAGAAACA
CGTGATATAAAACTACAAGGTCCTATTATTACCTAGGAT
AACTTTCGCCTCTATCTATGTCTCCTGTTGCTGGTTGACC
CCAATCATTGAAACTGTATTCTTTGGAATGCTCAGCAGTC
TCCTCCAAGCTGCCATTCCCTCCTTATCTAGGTGGGCCCA
GTATTGGCTCAGGCTTCTTGGTTGTGCCACTTCCTAAGGC
TTGCAAATGCATTCAG

The present invention is not limited to these particular sequences, however, and may include CD44 regulatory regions having at least 70% homology, 80% homology, 90% homology or 99% homology to any of SEQ ID NOS: 1-3 and SEQ ID NOS: 89-91. To this end, the CD44 regulatory regions of the present invention may include any variant, natural or synthetic, that exhibits the properties of the CD44 regulatory regions that are discussed herein. Fragments of the CD44 regulatory regions that contain transcription factor binding sites are specifically contemplated.

In certain aspects, the sequences of the present invention should include at least one AP-1 binding site and/or NFκB binding site. Binding sites for AP-1 and NFκB are set forth below in SEQ ID NOs.: 92-99 and SEQ ID NOs.: 100-101, respectively.

(SEQ ID NO.: 92)
AAACCCTGACTCACTGCCTCC
(SEQ ID NO.: 93)
CAGTGAGTCAGGG
(SEQ ID NO.: 94)
CCCTGACTCACTG
(SEQ ID NO.: 95)
AGGCAGTGAGTCAGGGTTTAC
(SEQ ID NO.: 96)
ATCCTGTGACTCAGCTTCTAT
(SEQ ID NO.: 97)
AGCTGAGTCACAG
(SEQ ID NO.: 98)
CTGTGACTCAGCT
(SEQ ID NO.: 99)
AGAAGCTGAGTCACAGGATGA
(SEQ ID NO.: 100)
CTGGGAAATCCCTTC
(SEQ ID NO.: 101)
AAGGGATTTCCCAGA

These binding sites are also not limiting to the present invention and may include homologues and conservative variants thereof, i.e. sequences having 70% homology, 80% homology, 90% homology or 99% homology to any of SEQ ID NOS: 92-101 or any variant that maintains AP-1 and/or NFκB binding affinity.

The isolated nucleic acids of the present invention may be substantially free from other nucleic acids. For most cloning purposes, DNA is a preferred, but non-limiting, nucleic acid. One or a combination of the foregoing sequences may be subcloned into an expression vector and subsequently transfected into a host cell of choice wherein the sequences result in expression of some downstream gene, as discussed in greater detail below. Such procedures may be used for a variety of utilities, such as those discussed in detail below, or, alternatively, to establish a cell line from which the regulatory mechanisms of the sequence may be studied or used.

Recombinant Vectors and Transfection Methods

In accordance with the foregoing, the present invention also relates to recombinant vectors and recombinant hosts, both prokaryotic and eukaryotic, which contain nucleic acid molecules encoding a gene where the CD44 regulatory regions of the present invention control expression of that gene. These nucleic acid molecules, in whole or in part, can be linked with other DNA molecules that are not naturally linked, to form “recombinant DNA molecules” which encode the targeted gene. These vectors may be comprised of DNA or RNA. For most cloning purposes DNA vectors are preferred. Typical vectors include plasmids, modified viruses, bacteriophage, cosmids, yeast artificial chromosomes, and other forms of episomal or integrated DNA. It is within the purview of the skilled artisan to determine an appropriate vector for a particular gene transfer, screening assay, or other use.

Methods of subcloning nucleic acid molecules of interest into expression vectors, transforming or transfecting host cells containing the vectors, and methods of making substantially pure protein comprising the steps of introducing the respective expression vector into a host cell, and cultivating the host cell under appropriate conditions are well known. Any known expression vector may be utilized to practice this portion of the invention, including any vector containing a suitable promoter and other appropriate transcription regulatory elements, inclusive of or outside of those discussed herein. The resulting expression construct is transferred into a prokaryotic or eukaryotic host cell to produce recombinant protein.

Expression vectors are defined herein as DNA sequences that are required for the transcription of cloned DNA and the translation of their mRNAs in an appropriate host. Such vectors can be used to express eukaryotic DNA in a variety of hosts such as, but not limited to, bacteria, blue green algae, plant cells, insect cells and animal cells.

An appropriately constructed expression vector may contain: an origin of replication for autonomous replication in host cells, selectable markers, a limited number of useful restriction enzyme sites, a potential for high copy number, and active promoters. A promoter is defined as a DNA sequence that directs RNA polymerase to bind to DNA and initiate RNA synthesis. A strong promoter is one which causes mRNAs to be initiated at high frequency. Techniques for such manipulations can be found described in Sambrook, et al. (1989, Molecular Cloning. A Laboratory Manual; Cold Spring Harbor Laboratory, Cold Spring Harbor, N.Y.) are well known and available to the artisan of ordinary skill in the art.

Commercially available mammalian expression vectors which may be suitable, include, but are not limited to, pCAGIG (Addgene), pcDNA3.neo (Invitrogen), pcDNA3.1 (Invitrogen), pCI-neo (Promega), pLITMUS28, pLITMUS29, pLITMUS38 and pLITMUS39 (New England Bioloabs), pcDNAI, pcDNAIanp (Invitrogen), pcDNA3 (Invitrogen), pMClneo (Stratagene), pXT1 (Stratagene), pSG5 (Stratagene), EBO pSV2-neo (ATCC 37593) pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146), pUCTag (ATCC 37460), and lZD35 (ATCC 37565).

Also, a variety of bacterial expression vectors are available, including but not limited to pCR2.1 (Invitrogen), pET1 la (Novagen), lambda gtl 1 (Invitrogen), and pKK223-3 (Pharmacia). In addition, a variety of fungal cell expression vectors may be used, including but not limited to pYES2 (Invitrogen) and Pichie expression vector (Invitrogen). Also, a variety of insect cell expression vectors may be used, including but are not limited to pBlueBacIII and pBlueBacHis2 (Invitrogen), and pAcG2T (Pharmingen).

Generally speaking, recombinant host cells may be prokaryotic or eukaryotic, including but not limited to, bacteria such as E. coli, fungal cells such as yeast, mammalian cells including, but not limited to, cell lines of bovine, porcine, monkey and rodent origin; and insect cells. Mammalian species which may be suitable, −26 include but are not limited to, L cells L-M(TK-) (ATCC CCL1.3), L cells L-M (ATCC CCL 1.2), Saos-2 (ATCC HTB-85), 293 (ATCC CRL1573), hek 293t, Raji (ATCC CCL 86), CV-1 (ATCC CCL 70), COS-1 (ATCC CRL1650), COS-7(ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-l (ATCC CCL 26), MRC-5 (ATCC CCL171), CPAE (ATCC CCL 209). In certain embodiments, the cell lines include mammalian cancer cells such as, but not limited to, MDA-MB-231, SUM159, SUM149, and MCF7.

In certain aspects, however, host cells may natively express the transcription factors AP-1 and/or NFκB or may be genetically engineered to express one or both of these transcription factors.

Drug Screening Assay

The CD44 regulatory sequences of the present invention may be used in one or more of any of a wide array of uses, including, but not limited to a drug screening assay. A cell line exhibiting high CD44 expression, such as SUM159, SUM 149 or MCF7, may be used as the basis for a drug screening assay. Alternatively, a cell line may be used that has been transfected with a vector in accordance with the foregoing methods. In certain embodiments, the vector includes a coding region or reporter gene and one or more of the CD44 regulatory regions of the present invention that regulate the expression of that gene in a cell. In one embodiment, the coding gene is a non-CD44 coding region.

Reporter gene vectors and well-known are widely used in the art. One non-limiting example of which includes the green fluorescent protein (GFP) reporter system. In this system, the vector contains a promoter region and expression of a green florescent protein (GFP). The CD44 regulatory regions are added such that they control expression of the GFP, upon transfection into a host cell or organism. Expression levels may be monitored using a variety of techniques, which are discussed in greater detail below.

The present invention is not limited to the use of a GFP reporter system, however, and other systems or proteins known in the art may be used such as, but not limited to, those associated with red fluorescent protein, yellow fluorescent protein (e.g., Living Colors™ Fluorescent Proteins from Clontech, Mountain View Calif.), beta-galactosidase, luciferase, and the like. Alternatively, any polypeptide sequences detectable by virtue of an activity (e.g., an enzymatic activity that can be measured), antigenicity (e.g., detectable immunologically), a radioactive, chemoluminescent or fluorescent label, or the like. Additional reporter systems or vectors using such reporter systems will be readily apparent to one of skill in the art.

In the screening assay, expression levels of the gene of interest, i.e. CD44, a reporter gene, and/or any gene associated with the CD44 regulatory region, are first measured to establish a baseline expression levels. One or more compounds or therapeutic agents may then be administered to the cell lines, and expression levels of the gene are re-measured to determine what effect, if any, the therapeutic agent had.

The therapeutic agents tested may be a non-proteinaceous organic or inorganic molecule, a peptide (e.g., as a potential prophylactic or therapeutic peptide vaccine), a protein, DNA (single or double stranded), RNA (such as siRNA or shRNA), or the like. It will become evident upon review of the disclosure and teachings of this specification that any such peptide or small molecule which effectively binds to the CD44 regulatory region and competes with AP-1 and/or NFκB for binding to the CD44 regulatory region or otherwise impedes the regulatory activity of the CD44 regulatory region, represents a possible lead therapeutic relating to prophylactic or therapeutic treatment of a disease state characterized by CD44 expression or overexpression, particularly carcinomias having a high CD44 expression profile. To this end, interaction assays may be utilized for the purpose of high throughput screening to identify compounds that occupy or interact with the CD44 regulatory regions of the present invention.

Various detection assays are known in the art may be used in accordance with the foregoing, including, but not limited to, an ELISA assay, a radioimmune assay, a Western blot analysis, flow cytometry, any homogenous assay relying on a detectable biological interaction not requiring separation or wash steps (e.g., see AlphaScreen from PerkinElmer) and/or SPR-based technology (e.g., see BIACore)). Compounds and/or therapeutic agent candidates identified through use of an the CD44 regulatory regions of the present application may be detected by a variety of assays. The assay may be a simple “yes/no” assay to determine whether there is a change in the ability to expression profile, or may be made quantitative in nature by utilizing an assay such as an ELISA based assay, a homogenous assay, or an SPR-based assay. To this end, the present invention relates to any such assay, regardless of the known methodology employed, which measures the ability of a test compound to affect the ability of the CD44 regulatory region to express the targeted gene.

In certain, non-limiting aspects, the present invention relates to a high content screening (HCS) assay for therapeutic agents targeting CD44 regulatory regions of the present invention. As is understood in the art, a HCS assay combines qualitative observations with quantitative measurements by integrating a cell-based assay (e.g., in a standard 96 or 384 well format) with high resolution fluorescence microscopy with automated image acquisition, specialized image processing algorithms for quantitative single cell analysis, and data and image archiving. It provides assessment (e.g., detection, distinction, and quantification) of individual cells or clusters of cells within an array of cells based on preselected parameters. Methods of HCS are known in the art. See, e.g., Ghosh and Haskins, “A Flexible Large-Scale Biology Software Module for Automated Quantitative Analysis of Cell Morphology” in Business Briefings: Future Drug Discovery 2004: 1-4.

Performing a screen on wide array of therapeutic agents requires parallel handling and processing of many compounds and assay component reagents. Standard high throughput screens use mixtures of compounds and biological reagents along with some indicator compound loaded into arrays of wells in standard microtiter plates with 96 or 384 wells. The signal measured from each well, either fluorescence emission, optical density, or radioactivity, integrates the signal from all the material in the well giving an overall population average of all the molecules in the well. In contrast to high throughput screens, high-content screens provide more detailed information about the temporal-spatial dynamics of cell constituents and processes, and how they are affected by potential drug candidates. High-content screens automate the extraction of multicolor fluorescence information derived from specific fluorescence-based reagents incorporated into cells (Giuliano and Taylor (1995), Cum Op. Cell Biol. 7:4; Giuliano et al. (1995) Ann. Rev. Biophys. Biomol. Struct. 24:405). Cells are analyzed using an optical system that can measure spatial, as well as temporal dynamics. (Farkas et al. (1993) Ann. Rev. Physiol. 55:785; Giuliano et al. (1990) In Optical Microscopy for Biology. B. Herman and K. Jacobson (eds.), pp. 543-557. Wiley-Liss, New York; Hahn et al (1992) Nature 359:736; Waggoner et al. (1996) Hum. Pathol. 27:494).

Such screening assays can be performed on living or fixed cells, using a variety of labeled reporter molecules, such as antibodies, biological ligands, nucleic acid hybridization probes, and multicolor luminescent indicators and “biosensors.” The choice of fixed or live cell screens depends on the specific cell-based assay required.

Fixed cell assays provide a simple approach because an array of initially living cells in a microtiter plate format can be treated with various agents and doses being tested, then the cells can be fixed, labeled with specific reagents, and measured. No environmental control of the cells is required after fixation. Spatial information is acquired, but only at one time point. The availability of thousands of antibodies, ligands and nucleic acid hybridization probes that can be applied to cells makes this an attractive approach for many types of cell-based screens. The fixation and labeling steps can be automated, allowing efficient processing of assays.

Live cell assays are more sophisticated and powerful, since an array of living cells containing the desired reagents can be screened over time, as well as space. Environmental control of the cells (temperature, humidity, and carbon dioxide) is required during measurement, since the physiological health of the cells must be maintained for multiple fluorescence measurements over time. There is a growing list of fluorescent physiological indicators and “biosensors” that can report changes in biochemical and molecular activities within cells (Giuliano et al., (1995) Ann. Rev. Biophys. Biomol. Struct. 24:405; Hahn et al., (1993) In Fluorescent and Luminescent Probes for Biological Activity. W. T. Mason, (ed.), pp. 349-359, Academic Press, San Diego).

The types of biochemical and molecular information accessible through fluorescence-based reagents applied to cells include ion concentrations, membrane potential, specific translocations, enzyme activities, gene expression, as well as the presence, amounts and patterns of metabolites, proteins, lipids, carbohydrates, and nucleic acid sequences (DeBiasio et al., (1996) Mol. Biol. Cell. 7:1259; Giuliano et al., (1995) Ann. Rev. Biophys. Biomol. Struct. 24:405; Heim and Tsien, (1996) Curr. Biol. 6:178).

The present invention is not necessarily limited to the foregoing and one of skill in the art would readily appreciate additional uses and methods of using the CD44 regulatory regions identified herein.

The following are examples supporting the foregoing invention. They are not to be construed as limiting to the invention.

### EXAMPLES

### Materials and Methods

A. Computational Prediction of CD44 Cis-Regulatory Elements

Multiple sequence alignment methods were used to identify evolutionarily conserved noncoding DNA sequences as putative gene regulatory elements. The sequences and annotations of analyzed genes along with their homologs from the various genomes were retrieved using noncoding sequence retrieval system, NCSRS. These sequences were then aligned using multi-LAGAN to identify elements with >70% identity over a 100 bp span to ensure significance in sequence conservation. The percent identity and length of the CR were used to calculate a score for each conserved region (CR) (score=percent identity+(length/60)).

B. Cell Culture

The breast cancer cell lines SUM159 cells (Asterand Inc. Detroit, Mich.), MDA-MB-231 cells (ATCC), MCF7 cells (gift from Dr. Nanjoo Suh at Rutgers University) were cultured according to the guidelines from the suppliers. All cell lines were maintained at 37° C. in a humidified incubator with 5% CO₂.

C. Reporter Plasmids

Conserved regions were amplified by PCR from mouse genomic DNA (Table 1), subcloned into a GFP reporter plasmid with a basal beta-globin promoter (βGP-GFP) and verified by sequencing.

TABLE 1
PCR Primers for the amplification of the three conserved regions.

| | PCR | | | |
|---|---|---|---|---|
| Conserved | product | | | |
| Region | length (bp) | Primer | Mouse Sequence | Human Sequence |
| CD44CR1 | 829 | Forward | GGGCAGGATGAGTGGTTATTGAGA | GGTGAAATGCCCTATAGCTCAACTCTG |
| | | | (SEQ ID NO.: 4) | (SEQ ID NO.: 5) |
| | | Reverse | GGGTGGAATACAACCACACTGCAT | GTGCTTATTTCACATTGCATTCCTGC |
| | | | (SEQ ID NO.: 6) | (SEQ ID NO.: 7) |
| CD44CR2 | 735 | Forward | CACTGTTTGAAATGGGTGGCGATG | TGCTGCAATATAGACTTTCTGACC |
| | | | (SEQ ID NO.: 8) | (SEQ ID NO.: 9) |
| | | Reverse | GCATGAAACCACAGAGCCTACAGA | GACTGTCGTGTTTGTTCTCACTC |
| | | | (SEQ ID NO.: 10) | (SEQ ID NO.: 11) |
| CD44CR3 | 732 | Forward | TCCTACCTGTCTCCAGTGTTGTGA | TGGGCCCAGCTCAGTTTATACCTT |
| | | | (SEQ ID NO.: 12) | (SEQ ID NO.: 13) |
| | | Reverse | AACAACATTCCACAGACTGGCTCG | GGTCCCTTCTTCCCATCAGTTTCT |
| | | | (SEQ ID NO.: 14) | (SEQ ID NO.: 15) |

D. Transfection

For transfections, cells were seeded onto poly-L-Lysine (PLL) treated coverslips in 24 well plates. Cells were transfected with Lipofectamine LTX (Invitrogen), per the manufacturer's recommendations. Following a 24 hour incubation period, nuclei were stained with Hoechst33342 (Sigma). Cells were then fixed with 4% paraformaldehyde in PBS for 12 minutes at room temperature, stained with anti-GFP (Invitrogen) for 2 hours, and followed with Dylight 488 (Jackson Immuno) secondary antibody. Coverslips were adhered to slides with Fluoro-Gel (Electron Microscopy Sciences). GFP-expres sing cells were visualized by a Zeiss Axiolmager A1 fluorescence microscopy.

E. qRT-PCR

RNA was isolated from cells using Tri Reagent (Ambion). cDNA was prepared by reverse transcription using the qScript cDNA SuperMix (Quanta), and used as a template for RT-PCR (PerfeCTa SYBR Green FastMix (Quanta)). RT-PCR reaction was run on a Roche LightCycler using primer sequences obtained from the Harvard Primer Bank (Table 2). Threshold cycles were normalized relative to GAPDH expression.

TABLE 2
qPCR primer sequences obtained from Harvard Primer Bank

| Name | Primer | Sequence |
|---|---|---|
| CD44 | Forward | TGCCGCTTTGCAGGTGTATT (SEQ ID NO: 16) |
| | Reverse | CCGATGCTCAGAGCTTTCTCC (SEQ ID NO.: 17) |
| CD24 | Forward | CTCCTACCCACGCAGATTTATTC (SEQ ID NO.: 18) |
| | Reverse | AGAGTGAGACCACGAAGAGAC (SEQ ID NO.: 19) |
| GAPDH | Forward | CATGAGAAGTATGACAACAGCCT (SEQ ID NO.: 20) |
| | Reverse | AGTCCTTCCACGATACCAAAGT (SEQ ID NO.: 21) |

F. Immunocytochemistry

For immunocytochemistry, cells were plated on PLL treated coverslips and incubated for 24 hours and then fixed to coverslips using 4% paraformaldehyde, blocked with 10% Donkey Serum (Jackson Immunology) and then incubated with the primary antibody for 2 hrs at room temp. The following antibodies were used [CD44 (Chemicon); CD24 (Santa Cruz); NFκB-c-Rel (Chemicon); NFκB-p50 (Upstate); NFκB-p65 (Abcam); Fra1 (Santa Cruz); Fra-2 (Santa Cruz); cFos (Santa Cruz); cJun(N) (Santa Cruz); cJun(D) (Santa Cruz); JunB (Santa Cruz); FosB (Santa Cruz)]. Following primary incubation, cells were incubated with a fluorescent secondary antibody (Jackson Immunology). Nuclei were stained with Hoechst33342.

G. Genomic DNA Sequencing

Genomic DNA was collected from the human cell lines using the Promega Genomic DNA kit as per manufacturer's recommendations. Genomic DNA from each cell line was sequenced using primers specific for the conserved regions (Table 1, above). Genomic DNA was aligned using the online program ClustalW.

H. Electrophoresis Mobility Shift Assay and Supershift

Single stranded DNA probes were designed from mouse CD44CR1 and labeled with the 3′ Biotin End Labeling Kit (Thermo Scientific) as per manufacturer's suggestions. Nuclear extracts were collected from each breast cancer cell line using NE-PER nuclear and cytoplasmic extraction reagents (Thermo Scientific). Binding reactions were performed and detected using the LightShift Chemiluminescent EMSA kit (Thermo Scientific) per manufacturer's recommendations. DNA-protein complexes were run on 10% non-denaturing poly-acrylamide gels and transferred onto Biodyne Plus membrane (Pall). Membranes were cross-linked in a UV imager for 15 minutes. EMSA probe sequences are in Table 3. Supershift assays were performed in a similar fashion. Antibodies were added to select reactions 15 minutes prior to addition of labeled probes.

TABLE 3
EMSA Probes

| EMSA | |
|---|---|
| Probes | Forward Sequence |
| CD44CR- | GATTGCCAACACCCAGGAAATAAGGAAGAATGAGACAGAAACCAGATGTGTTG |
| 60-170 | GTGTCATCCTGTGACTCAGCTTCTATTCTGGTTGCTGATAAATAAAGAAGAGTTT |
| | CCA (SEQ ID NO.: 22) |
| CD44CR1- | CTGAGGGCAGTAAACCCTGACTCACTGCCTCCTTCCTACCACAGTTTCCAAAAC |
| 600-660 | ACTGCTA(SEQ ID NO.: 23) |
| CD44CR1- | ATTGCGCCCTTGTCTCTATGCAGATCTCAGTCAGTCTGGGCCACCATGTATGCA |
| 660-745 | AACAGCTCTTT (SEQ ID NO.: 24) |
| CD44CR1- | CTGAGGGCAGTAAACCCTGACTCACTGCCTCCTTCCTACCACAGTTTCCAAAAC |
| 600-745 | ACTGCTATTGCGCCCTTGTCTCTATGCAGATCTCAGTCAGTCTGGGCCACCATG |
| | TATGCAAACAGCTCTTTCTGGGAAATCCCTTCT (SEQ ID NO.: 25) |
| CD44CR1- | CCAGTGGGTTTCCCCACCTTTCCTTCACTCACATCTCTCTCTCCCC(SEQ ID |
| 450-495 | NO.: 26) |
| CD44CR1- | CTCCCCGACTTTCTTCTTCGAAGTTCCCATAGGCCA(SEQ ID NO.: 27) |
| 490-525 | |
| CD44CR1- | CATGCATGTACAGACTTCGTCCGAAGCCTCCCTGTGAGCA(SEQ ID NO.: 28) |
| 550-590 | |
| CD44CR1- | TCATCCTGTGACTCAGCTTCTATT(SEQ ID NO.: 29) |
| AP-1-1 | |
| CD44CR1- | GTAAACCCTGACTCACTGCCTCCT(SEQ ID NO.: 30) |
| AP-1-2 | |
| CD44CR1- | CTCTTTCTGGGAAATCCCTTCTTGT(SEQ ID NO.: 31) |
| NFkB | |
| CD44CR1- | AACACCCAGGAAATAAGGAAGAATGAGAC(SEQ ID NO.: 32) |
| ETS-1 | |
| CD44CR1- | GTTGGTGTCATCCTGTGACTC(SEQ ID NO.: 33) |
| ETS-2 | |

I. Site Directed Mutagenesis

Site directed mutagenesis was performed as previously described using primer sequences as listed in Table 4. Treated DNA was transformed into NEB5α cells (NEB) and plated onto LB-amp plates. Constructs were collected by Qiagen midi-prep and then sequenced to verify the resulting mutation. Mutated constructs were transfected into cells and tested for GFP expression.

TABLE 4
Primers used for site directed mutagenesis.

| Name | Primer | Sequence |
|---|---|---|
| CD44CR1ΔAP-1-1 | Forward | GGTGTCATCCTGTGAGCTTCTATTCTGG(SEQ ID NO.: 34) |
| | Reverse | CCAGAATAGAAGCTCACAGGATGACACC(SEQ ID NO.: 35) |
| CD44CR1ΔAP-1-2 | Forward | GGCAGTAAACCCTCACTGCCTCCTTCCTACC(SEQ ID NO.: 36) |
| | Reverse | GGTAGGAAGGAGGCAGTGAGGGTTTACTGCC(SEQ ID |
| | | NO.: 37) |
| CD44CR1ΔNFkB | Forward | CAAACAGCTCTTTCTAATCCCTTCTTGTC(SEQ ID NO.: 38) |
| | Reverse | GACAAGAAGGGATTAGAAAGAGCTGTTTG(SEQ ID NO.: 39) |
| SDM Control Deletion | Forward | CCATGGGCTTTCCACATGGTAAATGTCCCTTTGC |
| | | (SEQ ID NO.: 40) |
| | Reverse | GCAAAGGGACATTTACCATGTGGAAAGCCCATG |
| | | (SEQ ID NO.: 41) |

J. Chromatin Immunoprecipitation

Chromatin immunoprecipitation (ChIP) was performed as previously described. Sonication was performed using a Branson 450 Digital Sonicator. The chromatin extract was pre-cleared with protein A beads (NEB). Protein-DNA crosslinks were reversed with 30 μl 5M NaCl and incubating samples at 65° C. for 4 hours. Proteins were digested with 0.1 mM EDTA, 20 mM Tris-HCl and 2 μl Proteinase K solution (Active Motif) for 2 hrs at 42° C. DNA was purified using phenol-chloroform extraction. PCR was performed using primers to identify DNA:protein interactions (Table 5). Rabbit IgG and anti-GFP antibody served as negative control.

TABLE 5
Primers used for ChIP
ChIP Probes CD44CR-AP1-1-373 bp

| | |
|---|---|
| CD44CR1-AP1-1 Forward | AGGTGAGCGGATATCAACCAAGGA(SEQ ID NO.: 42) |
| CD44CR1-AP1-1 Reverse | AGAACTCAGTGCCGTGTCGATAGT(SEQ ID NO.: 43) |

ChIP Probes CD44CR1-NFkB-362 bp

| | |
|---|---|
| CD44CR1-NFkB Forward | CCAGGTATGCTATGTTTGGTTAAGCCC(SEQ ID NO.: 44) |
| CD44CR1-NFkB Forward | GTGGAGTTGGAAAGACAGATTGGC(SEQ ID NO.: 45) |

ChIP Probes CD44CR1-AP1-2-400 bp

| | |
|---|---|
| CD44CR1-AP1-1 Forward | TCTCTCCCACTGCTTTCCTCCAAA(SEQ ID NO.: 46) |
| CD44CR1-AP1-1 Reverse | GTGCTTATTTCACATTGCATTCCTGC(SEQ ID NO.: 47) |

### Example 1

### Prediction of Cis-Regulatory Elements for CD44 Expression Using Sequence Alignment Analysis

To understand the molecular mechanism of CD44 expression in breast cancer cells, highly conserved regions of non-coding DNA were computationally predicted as cis-regulators of CD44 expression.

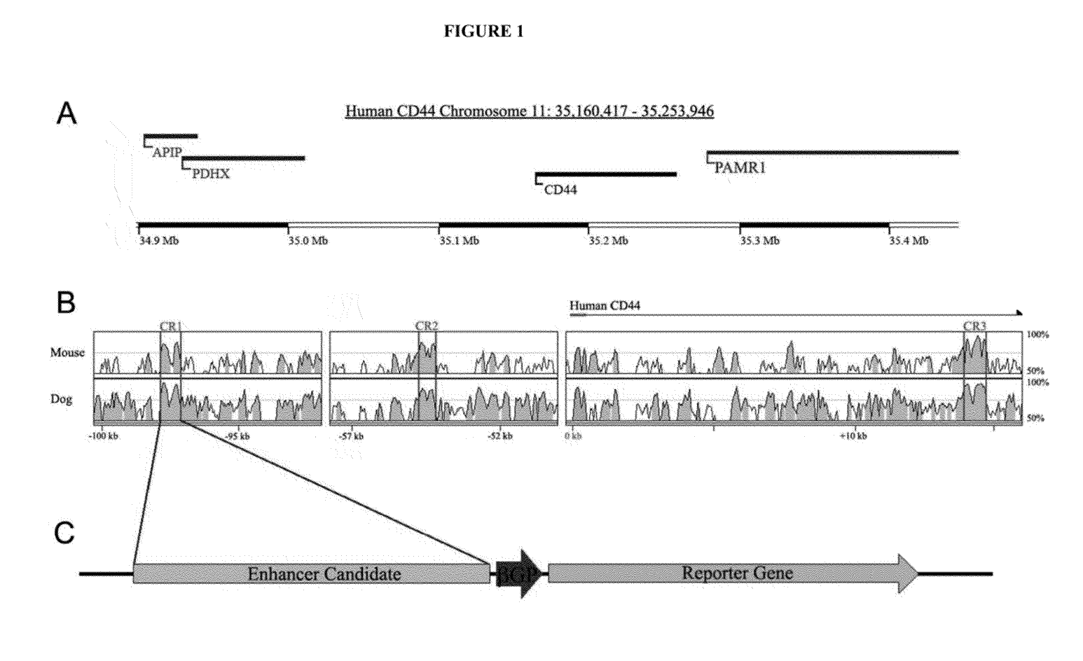

Multiple sequence alignment using the human CD44 genomic region as baseline revealed homologous regions in mouse, dog (FIG. 1A—illustrating a genomic map of the human CD44 and surrounding genes located on chromosome 11p3) and other mammalian species. A total of 14 conserved regions (CR) (>100 consecutive base pairs of sequence with >70% sequence identify) were identified.

FIG. 1B illustrates the multiple sequence alignment of homologous CD44 sequences and the 14 evolutionarily conserved regions. Conserved regions 1-3 (CR1-CR3) have the highest levels of conservation. Peaks surrounded by the bars are highly conserved regions that have at least 70% conservation among species. These three highest conserved regions (CR1-3) were chosen for further experimental verification. CR1 contains 717 bp (human), 715 bp (mouse) (SEQ ID NOS: 89 and 1, respectively) and located ˜95 kbp upstream of mouse CD44 transcription start site with 78% conservation. CR2 contains 727 bp (human); 611 bp (mouse) (SEQ ID NOS.: 90 and 2, respectively) with 76% conservation and is located 55 kbp upstream of CD44. CR3 contains 567 bp (human), 604 bp (mouse) (SEQ ID NOS.: 91 and 3, respectively) with 79% conservation and it is located in the first intron of the CD44 gene.

To test the CRs for their ability to direct gene expression, the CRs were PCR amplified from mouse genomic DNA and subcloned into an expression vector containing a β-globin minimal promoter (βGP) and green fluorescent protein (GFP) as the reporter gene (FIG. 1C). Mouse DNA was used to validate that evolutionarily conserved elements can function in different species.

### Example 2

### Conserved Regions have the Ability to Direct Reporter GFP Expression in Breast Cancer Cells

The ability of the conserved regions to direct gene expression was tested using three previously characterized human breast cancer cells, MDA-MB-231, SUM159, and MCF7, each with a different CD44/CD24 expression profile. These cells were derived from epithelial adenocarcinoma, anaplasitic carcinoma, and epithelial carcinoma, respectively. Both MDA-MB-231 and SUM159 cells contain increased levels of CD44 expression, moreover, SUM159 cells have been characterized with cancer stem cell like features. Thus, these cells provide different lines of validation.

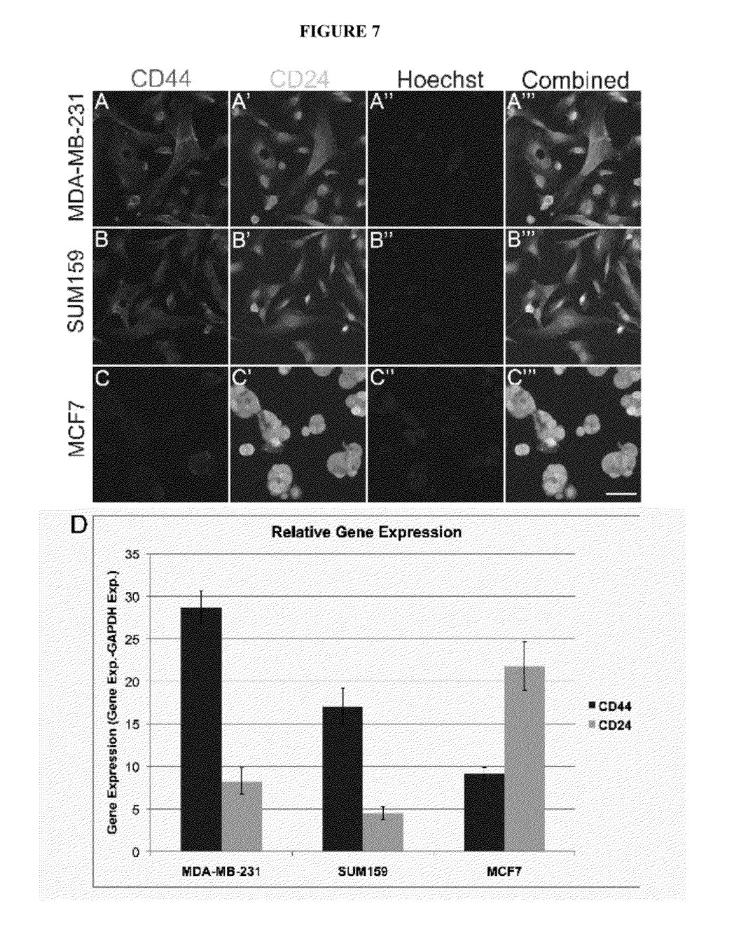

First, immunofluorescence staining was performed to verify CD44 and CD24 expression levels. Consistent with the genome-wide expression profiling study, MDA-MB-231 and SUM159 cells showed very high CD44 staining and low CD24 staining, while MCF7 showed low CD44 and high CD24 staining (FIGS. 7A-C).

Then, CD44 and CD24 expression level in the three cell lines was further quantified using quantitative PCR (qPCR). Results showed that MDA-MB-231 and SUM159 cells have the high CD44 and low CD24 expression, while MCF7 cells have the opposite expression profile, i.e., a higher CD24 and lower CD44 expression (FIG. 7D).

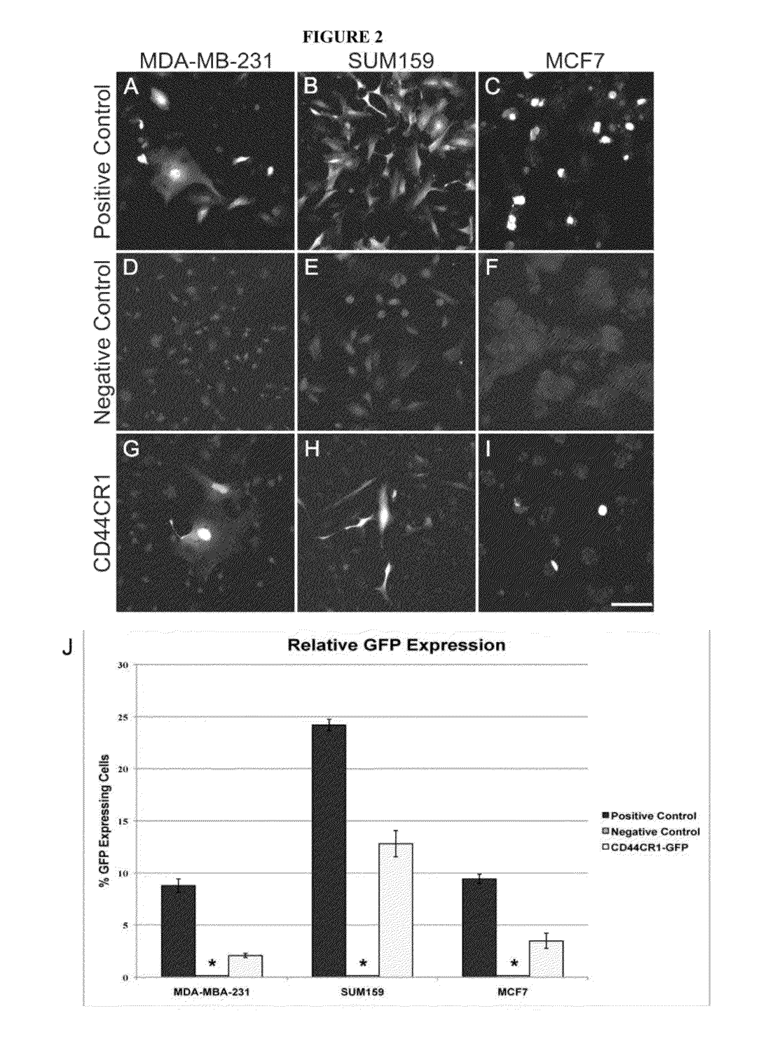

Next, each reporter construct containing one of the top three conserved regions of CD44 was individually tested by transfection into the three cell lines. Transfection of the positive control construct, CAG-GFP, resulted in positive expression of the reporter gene GFP (FIG. 2A-C) and demonstrated the ability of each of the cell lines to be transfected. As negative controls, a highly conserved region in Neurod1 and βGP alone, resulted in no visible GFP expression (FIG. 2D-F), indicating that not all highly conserved regions of genomic DNA nor βGP alone have the ability to direct gene expression. GFP expression was observed in all three cell lines after transfection with CD44CR1 constructs (FIG. 2G-I). However, different level of GFP expression among the three cell lines was observed (FIG. 2J—* represents no GFP-expressing cells were found). SUM159 had the highest percentage of GFP-expressing cells, followed by MDA-MB231 and MCF7. Transfection of constructs containing CD44CR2 and CD44CR3 also resulted in GFP-expressing cells (data not shown, under further investigation).

### Example 3

### Analysis of Trans-Acting Factor Binding Sites on the Conserved Regions of CD44

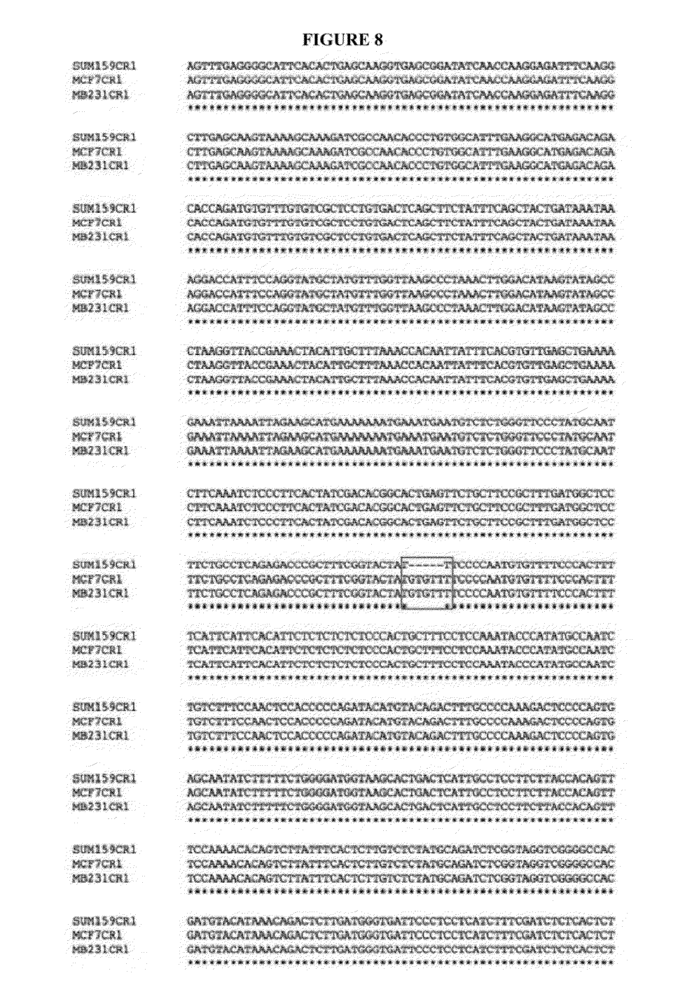

The ability of the conserved regions to direct different levels of reporter GFP expression among the three cell lines is most likely attributed to their interactions with trans-acting factors. Therefore, CR1-CR3 of both mouse and human were examined for trans-acting factor binding sites (TFBSs) and mutations in these sites. Genomic DNA of CR1-CR3 from each of the cell lines was collected and sequenced to determine if mutations in the region that disrupt TFBSs. Sequencing results show only a 4 bp span that differed between the three human cell lines in CR1 (FIG. 8). This 4 bp difference found in the SUM159 cells showed no disruption of key TFBSs. This indicates that the difference in GFP expression among these cells may not be associated with the DNA sequence. Thus, it is speculated that the difference in GFP expression may be the result of trans-acting factor binding in the cell lines. MatInspector (Genomatix, Germany) was used to identify putative TFBSs. Each conserved region resulted in over 150 putative TFBSs. These TFBSs were examined further for conservation between mouse and human sequences. CR1 contained the most TFBSs involved in breast cancer, cancer stem cells and embryonic development and therefore had the highest potential for regulating CD44 and for being involved in breast cancer (Table 6). The analysis was focused on the activities of CR1 in regulating gene expression in breast cancer cells.

TABLE 6
CD44CR1 transcription factor binding sites conserved between mouse and human.

| Family | Matrix | from-to | Str. | Sequence |
|---|---|---|---|---|
| V$HAND | V$PARAXIS.01 | 95-115 | (+) | cagaaACCAgatgtgttggtg (SEQ ID NO.: 48) |
| V$RP58 | V$RP58.01 | 99-111 | (−) | aacaCATCtggtt (SEQ ID NO.: 49) |
| V$RORA | V$REV-ERBA.02 | 115-137 | (−) | tagaagctgaGTCAcaggatgac (SEQ ID NO.: 50) |
| V$AP-1R | V$NFE2.01 | 116-136 | (−) | agaagCTGAgtcacaggatga (SEQ ID NO.: 51) |
| V$PBXC | V$PBX1_MEIS1.03 | 118-134 | (−) | aagctgagTCACaggat (SEQ ID NO.: 52) |
| V$AP-1R | V$TCF11 MAFG.01 | 118-138 | (+) | atcctgTGACtcagcttctat (SEQ ID NO.: 53) |
| V$AP-1F | V$AP-1.01 | 122-132 | (+) | tgtgACTCagc (SEQ ID NO.: 54) |
| V$AP-1F | V$AP-1.01 | 122-132 | (−) | gctgAGTCaca (SEQ ID NO.: 55) |
| V$GATA | V$GATA.01 | 145-157 | (+) | tgctGATAaataa (SEQ ID NO.: 56) |
| V$HOXC | V$PBX HOXA9.01 | 145-161 | (−) | ttctTTATttatcagca (SEQ ID NO.: 57) |
| V$PAX6 | V$PAX6.02 | 159-177 | (+) | gaagagtttCCAGgtatgc (SEQ ID NO.: 58) |
| V$BCL6 | V$BCL6.02 | 161-177 | (−) | gcataccTGGAaactct (SEQ ID NO.: 59) |
| V$STAT | V$STAT5.01 | 499-517 | (+) | tttcTTCTtcgaagttccc (SEQ ID NO.: 60) |
| V$CAAT | V$NFY.03 | 176-190 | (−) | taaaCCAAacatagc (SEQ ID NO.: 61) |
| V$NKXH | V$NKX31.01 | 203-217 | (+) | gacagtAAGTatacc (SEQ ID NO.: 62) |
| V$SNAP | V$PSE.02 | 212-230 | (+) | tatacCCTAaagttaccaa (SEQ ID NO.: 63) |
| V$HAML | V$AML3.01 | 241-255 | (−) | ggttGTGGttcagag (SEQ ID NO.: 64) |
| V$EBOX | V$MYCMAX.02 | 259-271 | (−) | tcaacaCATGtga (SEQ ID NO.: 65) |
| V$IRFF | V$IRF4.01 | 279-299 | (+) | aaaagaaaaaGAAAaaagaaa (SEQ ID NO.: 66) |
| V$IRFF | V$IRF7.01 | 292-312 | (+) | aaaaGAAAtgaaaattggaaa (SEQ ID NO.: 67) |
| V$OCT1 | V$OCT1.06 | 296-310 | (+) | gaaatgaaAATTgga (SEQ ID NO.: 68) |
| V$RBPF | V$RBPJK.02 | 508-522 | (−) | cctaTGGGaacttcg (SEQ ID NO.: 69) |
| V$YBXF | V$YB1.01 | 518-530 | (−) | cagatTGGCctat (SEQ ID NO.: 70) |
| V$CAAT | V$NFY.01 | 519-533 | (+) | taggCCAAtctgtct (SEQ ID NO.: 71) |
| V$SP1F | V$GC.01 | 537-551 | (−) | tgtggGGTGgggttg (SEQ ID NO.: 72) |
| V$CLOX | V$CDPCR3.01 | 585-607 | (−) | gccctcagaaaaagatATTGctc (SEQ ID NO.: 73) |
| V$AP-1R | V$BACH2.01 | 609-629 | (−) | aggcagTGAGtcagggtttac (SEQ ID NO.: 74) |
| V$AP-1R | V$NFE2.01 | 611-631 | (+) | aaaccCTGActcactgcctcc (SEQ ID NO.: 75) |
| V$CREB | V$TAXCREB.02 | 611-631 | (+) | aaacccTGACtcactgcctcc (SEQ ID NO.: 76) |
| V$CSEN | V$DREAM.01 | 612-622 | (−) | gaGTCAgggtt (SEQ ID NO.: 77) |
| V$AP-1F | V$AP-1.01 | 615-625 | (+) | cctgACTCact (SEQ ID NO.: 78) |
| V$AP-1F | V$AP-1.01 | 615-625 | (−) | agtgAGTCagg (SEQ ID NO.: 79) |
| V$CARE | V$CARF.01 | 626-636 | (+) | ggaagGAGGca (SEQ ID NO.: 80) |
| V$HAML | V$AML1.01 | 631-645 | (−) | aactGTGGtaggaag (SEQ ID NO.: 81) |
| V$AIRE | V$AIRE.01 | 631-657 | (−) | cagtgttttggaaactgTGGTaggaag (SEQ ID NO.: 82) |
| V$OCT1 | V$POU2F3.01 | 671-695 | (−) | tctATGCagatctcagt (SEQ ID NO.: 83) |
| V$OCT1 | V$OCT34.02 | 671-695 | (+) | gatctGCATagagacaa (SEQ ID NO.: 84) |
| V$FKHD | V$HNF3.01 | 703-719 | (−) | tgtatgcAAACagctct (SEQ ID NO.: 85) |
| V$NFKB | V$NFKAPPAB.01 | 725-737 | (+) | ctGGGAaatccct (SEQ ID NO.: 86) |
| V$NFKB | V$NFKAPPAB.01 | 726-738 | (−) | aaGGGAtttccca (SEQ ID NO.: 87) |
| V$EVI1 | V$EVI1.01 | 730-746 | (−) | aagacAAGAagggattt (SEQ ID NO.: 88) |

### Example 4

### Sequence Specific Trans-Acting Factor Binding with CD44CR1

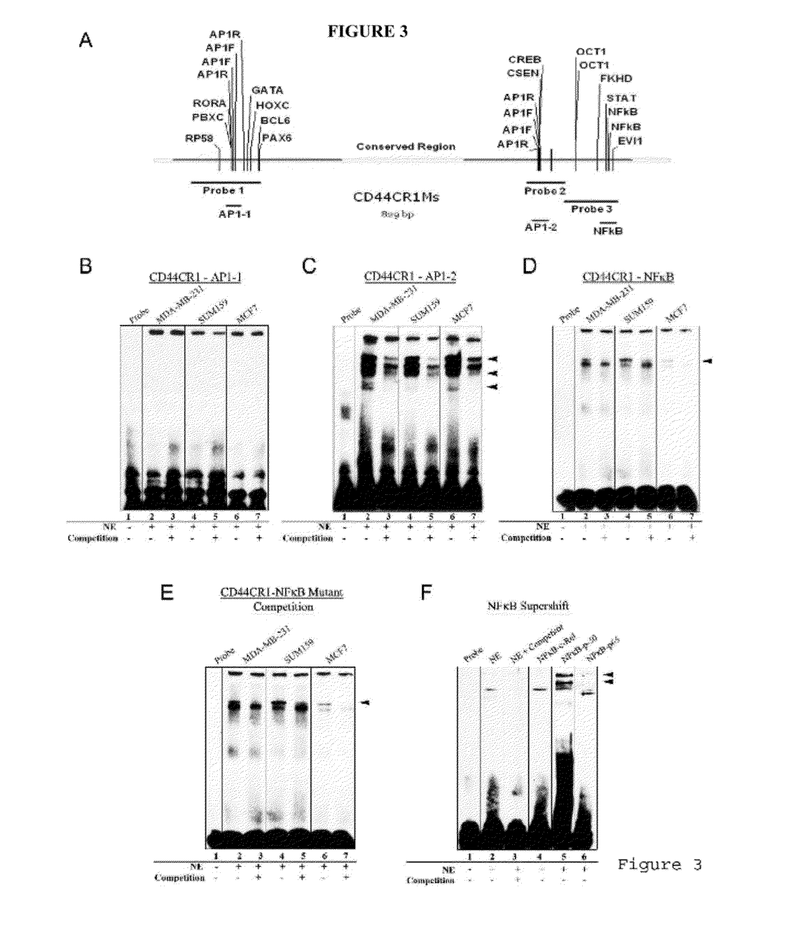

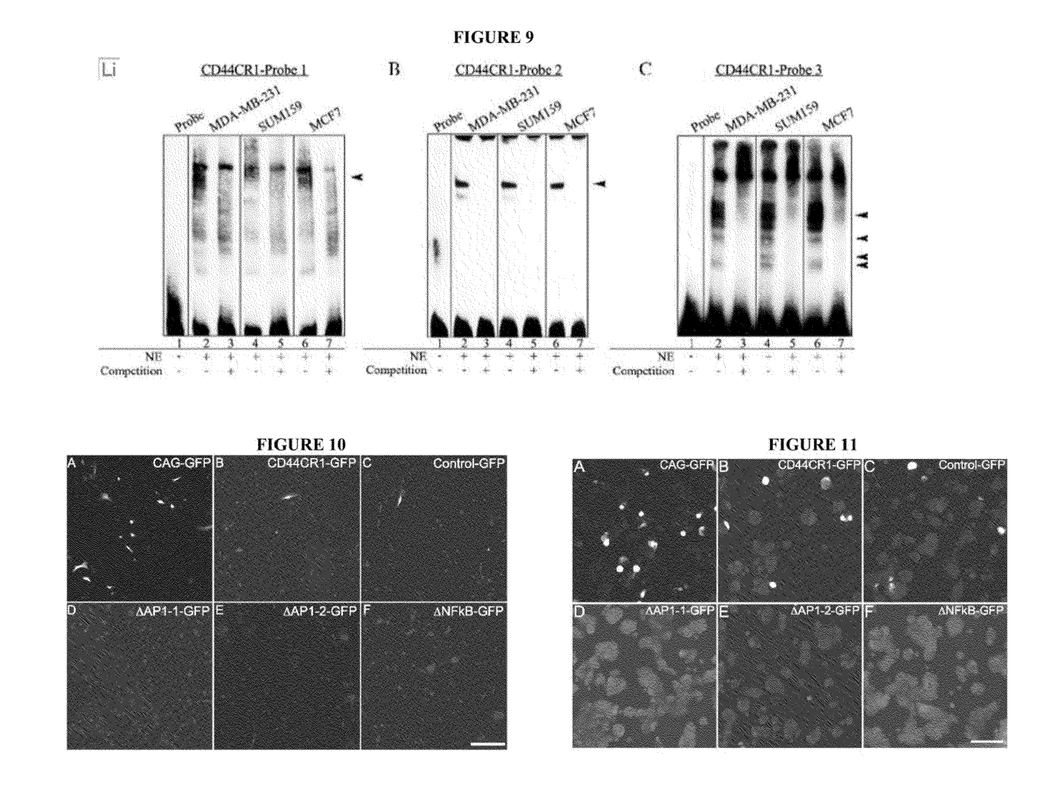

Electrophoretic mobility shift assays (EMSAs) were performed to determine if differences in GFP expression resulted from differences in TF binding in the cells. Double-stranded, biotin labeled oligonucleotides corresponding to regions of mouse CR1 were assayed for trans-acting factor binding using EMSA with nuclear extract from each of the cell lines (FIG. 3A). The shifted bands for three of the large probes spanning the length of the conserved regions in all three cell types (FIG. 9) indicating protein-DNA binding activity. Probe 1 (FIG. 9A) shows strong bands shifted with nuclear extracts from MDA-MB-231 and MCF7 cells only, while probe 2 (FIG. 3B) has a band shifted that is equally strong with all three cell lines. Probe 3 (FIG. 9C) shows a number of bands that can be competed away with an unlabeled probe. Although the bands in probe 3 are similar in all three cell lines, SUM159 does have a band that is not present in the other two cell lines (FIG. 9C).

Smaller probes were then used to narrow down regions of binding and to identify specific TFBSs. A probe designed to mimic the first AP-1 site (AP-1-1) showed no band shift (FIG. 3B), while the probe for the second AP-1 site (AP-1-2) showed a number of band shifts (FIG. 3C). Although these bands were not completely competed away, there was a significant reduction in band intensity with the addition of the competition probe. A probe for the region of NFκB binding also revealed band shifts. The intensity of the band differed among cell lines, with SUM159 showing the strongest shift (FIG. 3D).

To determine which specific proteins may bind with CD44CR1, we performed a mutant competition EMSA. Probes with the sequence mutated at the binding site for AP-1 and NFκB were used (Table 3). Mutant competition of AP-1-1 and -2 sites showed no shift (data not shown). However, mutant competition of NFκB did show a shift (FIG. 3E).

An EMSA supershift assay was performed to verify specific proteins binding using antibodies against NFκB proteins c-Rel, p50 and p65 (FIG. 3F). The antibody against NFκB-p50 was able to provide a significant shift in the labeled probe (FIG. 3F). NFκB-p65 showed a very faint shift similar to NFκB-p50 as well as a band that was downshifted. NFκB-c-Rel did not show a shift.

### Example 5

### Trans-Acting Factors AP-1 and NFκB Regulate CD44CR1-GFP Expression

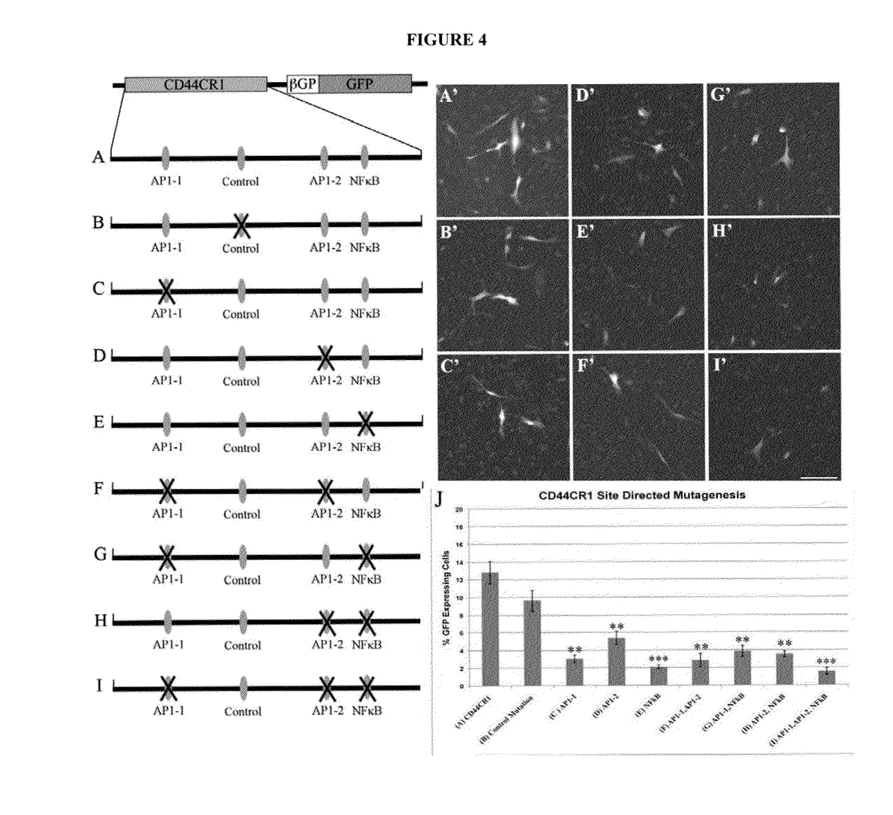

EMSA identified regions of CD44CR1 that were able to bind nuclear factors in each of the cell lines and the supershift assay was able to identify one specific protein, NFκB-p50, bound to this region. However, these in vitro assays are not sufficient to determine if these TFs have the ability to direct gene expression. To determine if the specific TFBSs are involved in the regulation of reporter GFP expression, site directed mutagenesis (SDM) was performed. The core binding sites for the two AP-1 TFBSs and NFκB binding site were deleted from the CD44CR1 reporter construct using SDM. Mutant constructs were transfected into each of the cell lines. Wild-type CR1 and a random mutation were used as control transfections. Results show that the control transfection did not result in a significant loss of GFP-expressing cells, whereas single site mutations at each AP-1 site and NFκB binding site (FIG. 4C-E) resulted in statistically significant decreases in GFP expression in SUM159 cells when compared to unmutated CR1 and the control mutation (FIG. 4A-B). (Control mutation at a non-conserved site (B′) showed no difference in GFP expression when compared to CD44CR1 (A′). Single site mutations of AP-1-1, AP-1-2 and NFκB (C′-E′) showed a significant reduction of GFP expression compared to CD44CR1. However, GFP expression was not eliminated entirely.)

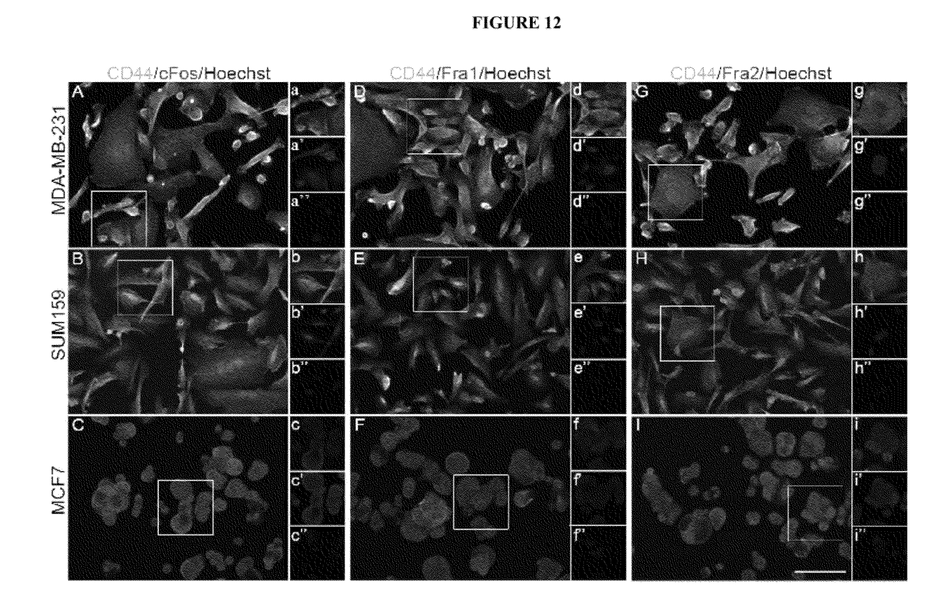

Since GFP expression was not completely abolished with the deletion of a single TFBS in SUM159, a combination of TFBSs were mutated (FIG. 4F-H). Results of transfections with combinatorial mutations again showed a statistically significant decrease in GFP expression (FIG. 4F-H). (Mutation of a combination of AP-1 and NFκB binding sites (F′-H′) did not reduce further GFP expression, however, the percentage of GFP expression was still significantly reduced compared to CD44CR1.) However, the percentage of GFP expression with two mutations did not change significantly as compared with single-mutation constructs. To determine whether all three sites are needed for CR1 to direct reporter expression, all three binding sites were mutated (FIG. 41). (Mutation of all three TFBSs (I′) showed the greatest reduction of GFP expression. **p=<0.0005 ***p=<1.0×10⁻⁵ (student's t-test). Scale bar=50 μM). The transfection of this construct showed very weak GFP expression. While in MDA-MB-231 and MCF7 cells, transfections of the mutant constructs resulted in no GFP-expressing cells (FIGS. 10 and 11).

### Example 6

### AP-1 and NFκB Expression Level Varies Among Breast Cancer Cell Lines

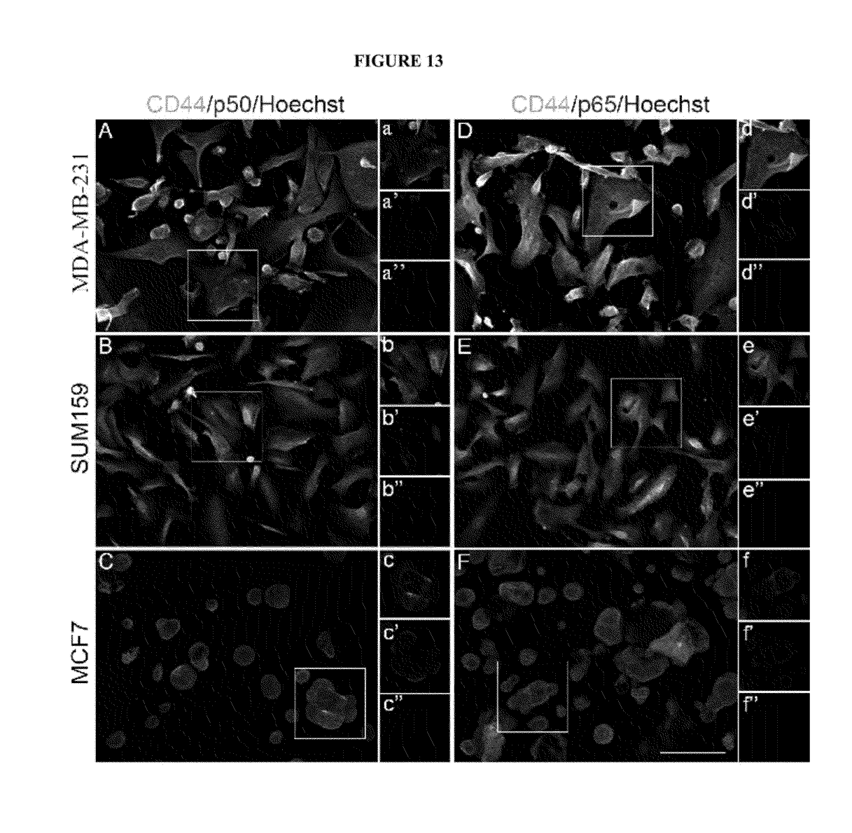

To further investigate the causes that lead to different GFP expression, immunocytochemistry was performed using antibodies against AP-1 and NFκB. For AP-1, antibodies against cJun, JunB, JunD, cFos, Fra1 and Fra2, components of AP-1 complex were tested with antibodies corresponding to CD44. (FIG. 12). Interestingly, all of these factors except JunB showed similar expression in all three cell lines (FIG. 5A-F). JunB staining was not detected in MDA-MB-231 cells (FIG. 5D). Similarly, NFκB expression was examined using antibodies against p50, p65, and cRel, components of NF-κB. Staining with antibodies against p50 and p65 (FIG. 13) showed similar expression in all three cell lines. However, cRel staining was not detected in MDA-MB-231 cells (FIG. 5G). Thus, AP-1 component JunB and NFκB component cRel may be major regulators of CD44CR1.

### Example 7

### Trans-Acting Factor Binding Patterns with CD44CR1 Differ Among Breast Cancer Cell Lines

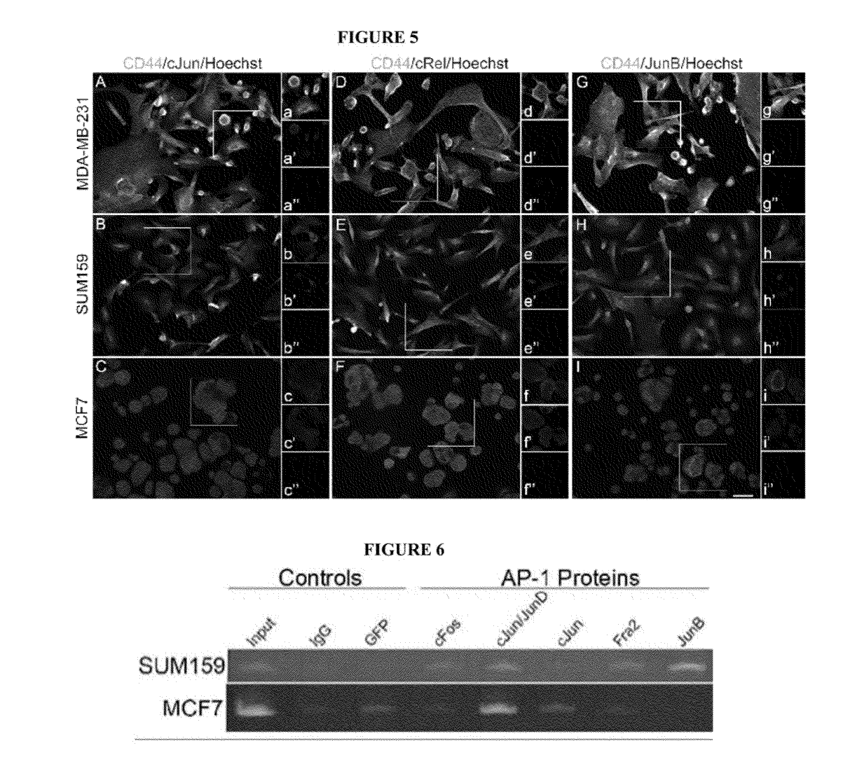

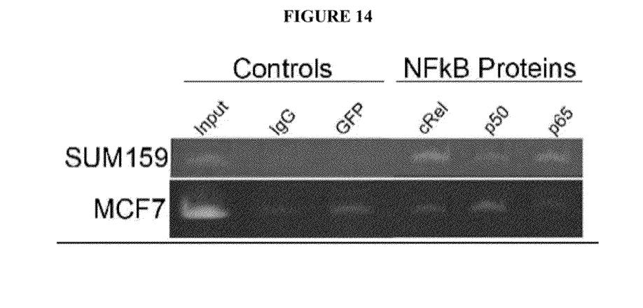

To determine whether the difference in reporter GFP expression among the three breast cancer cells is due to different trans-acting factor binding with CD44CR1, chromatin immuoprecipitation assays (ChIP) were performed using antibodies against individual components of AP-1 and NFκB. ChIP results show that in SUM159 cells only JunB bound with CD44CR1, while in MCF7 cells only JunD bound to CD44CR1 (FIG. 6). Although EMSA and supershift assays identified TFs NFκB-p50 and p65 (FIG. 3F), the ChIP results were inconclusive (FIG. 14).

What is claimed is:

1. A method for identifying a compound or therapeutic agent that inhibits CD44 expression in a cell, comprising:
providing a cell that expresses a gene using a CD44 regulatory region;
contacting the cell with a compound or therapeutic agent; and
detecting a change in expression level of the gene.

2. The method of claim 1, wherein the CD44 regulatory region comprises a sequence selected from the group consisting of SEQ ID NO.: 1 (CR1), SEQ ID NO.: 89 (CR1), SEQ ID NO.: 2 (CR2), SEQ ID NO.: 90 (CR2), SEQ ID NO.: 3 (CR3), SEQ ID NO.: 91 (CR3), combinations thereof, and variants thereof.

3. The method of claim 1, wherein the CD44 regulatory region comprises SEQ ID NO.: 1 (CR1), SEQ ID NO.: 89 (CR1) or a variant thereof.

4. The method of claim 1, wherein the CD44 regulatory region comprises a binding region selected from the group consisting of AP-1, NFκB, a combination thereof, and variants thereof.

5. The method of claim 4, wherein the AP-1 binding region comprises any one of SEQ ID NOS. 92-99, or a variant thereof.

6. The method of claim 4, wherein the NFκB binding region comprises any one of SEQ ID NOS.: 100-101, or a variant thereof.

7. The method of claim 1, wherein the gene is CD44.

8. The method of claim 1, wherein the gene is expressed from a vector that has been transfected into the cell.

9. The method of claim 1, wherein the gene is a reporter gene.

10. The method of claim 9, wherein the gene encodes a protein selected from the group consisting of green fluorescent protein, red fluorescent protein, yellow fluorescent protein, beta-galactosidase, luciferase, and combinations thereof.

11. The method of claim 1, wherein the detecting step comprises an ELISA assay, a radioimmune assay, a Western blot analysis, flow cytometry, or a high content screening assay.

12. A vector comprising:
a gene;
a promoter region; and
a non-coding CD44 regulatory region that controls expression of the gene.

13. The vector of claim 12, wherein the non-coding CD44 regulatory region comprises a sequence selected from the group consisting of SEQ ID NO.: 1 (CR1), SEQ ID NO.: 89 (CR1), SEQ ID NO.: 2 (CR2), SEQ ID NO.: 90 (CR2), SEQ ID NO.: 3 (CR3), SEQ ID NO.: 91 (CR3), combinations thereof, and variants thereof.

14. The vector of claim 12, wherein the non-coding CD44 regulatory region comprises SEQ ID NO.: 1 (CR1), SEQ ID NO.: 89 (CR1) or a variant thereof.

15. The vector of claim 12, wherein the non-coding CD44 regulatory region comprises a binding region selected from the group consisting of AP-1, NFκB, a combination thereof, and variants thereof.

16. The vector of claim 15, wherein the AP-1 binding region comprises any one of SEQ ID NOS. 92-99, or a variant thereof.

17. The vector of claim 15, wherein the NFκB binding region comprises any one of SEQ ID NOS.: 100-101, or a variant thereof.

18. The vector of claim 12, wherein the gene comprises CD44.

19. The vector of claim 12, wherein the gene is a reporter gene.

20. The vector of claim 12, wherein the gene encodes a protein selected from the group consisting of green fluorescent protein, red fluorescent protein, yellow fluorescent protein, beta-galactosidase, luciferase, and combinations thereof.

21. A kit for identifying a compound or therapeutic agent that inhibits CD44 expression in a cell, comprising:
a vector comprising a reporter gene; a promoter region; and a non-coding CD44 regulatory region that controls expression of the reporter gene; and
a reagent for detecting a product of the reporter gene.
